(19) United States
(12) Patent Application Publication
(10) Pub. No.: US 2009/0171254 A1
(43) Pub. Date: Jul. 2, 2009

(54) Time-reversal ultrasound focusing

(76) Inventors: Leonid Kushculey, (US); Vladimir Goland, Ashdod (IL)

Correspondence Address: EMPK & Shiloh, LLP;c/o Landon IP, Inc., 1700 Diagonal Road, Suite 450, Alexandria, VA, 22314, US

(21) Appl. No.: 12/003,811

(22) Filed: Jan. 2, 2008

(51) Int. Cl. A61N 7/02

(52) U.S. Cl. 601/3

(57) ABSTRACT

A method for creating a time reversed signal adapted to destroy a soft tissue, the method comprising emitting a first ultrasonic signal from a transmitter towards a tissue simulating medium which simulates said soft tissue, wherein the first ultrasonic signal has a first frequency characteristic; receiving the first ultrasonic signal in a receiver and converting the first ultrasonic signal to an electrical signal; converting the electrical signal to a digital signal; and time-reversing the digital signal to produce the time-reversed signal.

### FIELD OF THE DISCLOSURE

Embodiments of the disclosure relate to a high-intensity focused ultrasound (HIFU) system adapted to perform a non-invasive body contouring procedure.

### BACKGROUND

Ultrasound is a term used to describe sound waves having a frequency greater than the typical upper limit of human hearing, which is around 20 kilohertz (kHz).

Ultrasound is widely used in the medical field, both in diagnostics and in treatment. For example, ultrasound scanners are often used, in a method called “sonography”, for diagnosing certain medical conditions such as tumors and renal stones, and for monitoring fetus development during pregnancy. Therapeutic ultrasound is used for ablation and/or destroying of pathogenic objects and various tissues. Ultrasound may also be used to destroy fat tissues, for example in non-invasive body contouring procedures. The non-invasive body contouring is based on the application of focused therapeutic ultrasound that selectively targets and disrupts fat cells essentially without damaging neighboring structures. This may be achieved by, for example, a device, such as a transducer, that delivers focused ultrasound energy to the subcutaneous fat layer. Tissue destruction may be performed using high-intensity focused ultrasound (HIFU) energy, which can cause tissue damage by two main mechanisms—thermal and mechanical.

The thermal mechanism includes an increase of temperature within the treated area, obtained by a direct absorption of ultrasonic energy by the treated tissue. The increased temperature causes damaging processes, such as coagulation, within the tissue. The mechanical mechanism mainly includes streaming, shear forces, tension and cavitation, which is the formation of small bubbles within the tissue. These processes cause fractionation, rapture and/or liquefaction of cells, which in turn results in tissue destruction. Other destructive mechanisms, such as cell apoptosis, may also directly or indirectly be involved in the non-invasive ultrasonic treatment.

### SUMMARY

There is provided, in accordance with an embodiment of the disclosure, a method for creating a time reversed signal adapted to destroy a soft tissue, the method comprising emitting a first ultrasonic signal from a transmitter towards a tissue simulating medium which simulates said soft tissue, wherein the first ultrasonic signal has a first frequency characteristic; receiving the first ultrasonic signal in a receiver and converting the first ultrasonic signal to an electrical signal; converting the electrical signal to a digital signal; and time-reversing the digital signal to produce the time-reversed signal.

Optionally, the transmitter is a transducer unit comprising at least one transducer attached to at least one resonator, and the receiver is a sensor embedded in the tissue simulating medium. The method may fuher comprise emitting a second ultrasonic signal from the transducer unit towards said tissue simulating medium, wherein the second ultrasonic signal has a second frequency characteristic which is different than the first frequency characteristic of the first ultrasonic signal; receiving the second ultrasonic signal in the receiver and converting the second ultrasonic signal to a second electrical signal; converting the second electrical signal to a second digital signal; and time-reversing the second digital signal to produce a second time-reversed signal. Alternatively, the method may fuirther comprise emitting a second ultrasonic signal from a second transducer unit towards the tissue simulating medium, wherein the second ultrasonic signal has a second frequency characteristic which is different than the first frequency characteristic of the first ultrasonic signal; receiving the second ultrasonic signal in the receiver and converting the second ultrasonic signal to a second electrical signal; converting the second electrical signal to a second digital signal; and time-reversing the second digital signal to produce a second time-reversed signal.

Optionally, the transmitter is a sensor embedded in the tissue simulating medium and the receiver is a transducer unit comprising at least one transducer attached to at least one resonator. The method may further comprise emitting a second ultrasonic signal from the sensor towards the tissue simulating medium, wherein the second ultrasonic signal has a second frequency characteristic which is different than the first frequency characteristic of the first ultrasonic signal; receiving the second ultrasonic signal in the transducer unit and converting the second ultrasonic signal to a second electrical signal; converting the second electrical signal to a second digital signal; and time-reversing the second digital signal to produce a second time-reversed signal. Alternatively, the method may further comprise emitting a second ultrasonic signal from the sensor towards the tissue simulating medium, wherein the second ultrasonic signal has a second frequency characteristic which is different than the first frequency characteristic of the first ultrasonic signal; receiving the second ultrasonic signal in a second transducer unit and converting the second ultrasonic signal to a second electrical signal; converting the second electrical signal to a second digital signal; and time-reversing the second digital signal to produce a second time-reversed signal.

Optionally, the soft tissue is an adipose tissue.

Optionally, the method further comprises storing the time-reversed signal in a memory, along with a corresponding datum pertaining to a relative location of the transmitter and the receiver.

Optionally, the method further comprises converting the digital signal to a 1-bit signal.

Optionally, the method further comprises converting the time-reversed signal to a 1-bit signal.

There is further provided, in accordance with an embodiment of the disclosure, a system adapted to produce a time-reversed signal for destroying a soft tissue, the system comprising a transmitter adapted to emit an ultrasonic signal towards a tissue simulating medium which simulates the soft tissue; a receiver adapted to receive said ultrasonic signal and to convert said ultrasonic signal to an electrical signal; an analog-to-digital converter adapted to convert said electrical signal to a digital signal; and a signal processor adapted to time-reverse said digital signal and to produce said time-reversed signal.

Optionally, said transmitter is a transducer unit comprising at least one transducer attached to at least one resonator, and said receiver is a sensor embedded in said tissue simulating medium. The at least one transducer optionally comprises two or more transducers, each having a different resonant frequency.

Optionally, said transmitter is a sensor embedded in said tissue simulating medium and said receiver is a transducer unit comprising at least one transducer attached to at least one resonator. The at least one transducer optionally comprises two or more transducers, each having a different resonant frequency.

Optionally, the soft tissue is an adipose tissue.

Optionally, the system further comprises a memory adapted to store said time-reverse derived signal, along with a corresponding datum pertaining to a relative location of the transmitter and the receiver.

Optionally, said time-reversed signal is a 1-bit signal.

There is further provided, in accordance with an embodiment of the disclosure, a method for destroying a soft tissue within a focal area, the method comprising emitting a first time-reverse derived ultrasonic signal focused on a focal point within the focal area, wherein said time-reverse derived ultrasonic signal has a first frequency characteristic.

Optionally, the first time-reverse derived ultrasonic signal is adapted to induce cavitation within the focal area. _Optionally, the first time-reverse derived ultrasonic signal corresponds to a signal emitted by a transducer and received by a sensor embedded in a tissue simulating medium.

Optionally, the first time-reverse derived ultrasonic signal corresponds to a signal emitted by a sensor embedded in a tissue simulating medium and received by a transducer.

Optionally, the soft tissue is an adipose tissue.

Optionally, the first time-reverse derived ultrasonic signal is based on a 1-bit signal.

Optionally, the method further comprises emitting a second time-reverse derived ultrasonic signal which temporally overlaps the first time-reverse derived ultrasonic signal and is focused on the focal point, wherein the second time-reverse derived ultrasonic signal has a second frequency characteristic which is different than the first frequency characteristic of the first time-reverse derived ultrasonic signal.

There is further provided, in accordance with an embodiment of the disclosure, a device adapted to destroy a soft tissue within a focal area, the device comprising a transducer unit adapted to emit a first time-reverse derived ultrasonic signal having a first frequency characteristic, wherein the first time-reverse derived ultrasonic signal is adapted to be focused on a focal point within said focal area.

Optionally, the soft tissue is an adipose tissue.

Optionally, the first time-reverse derived ultrasonic signal corresponds to a signal received by a sensor embedded in a tissue simulating medium which simulates the soft tissue.

Optionally, the first time-reverse derived ultrasonic signal corresponds to a signal received by a transducer.

Optionally, the first time-reverse derived ultrasonic signal is adapted to induce cavitation within the focal area.

Optionally, the device further comprises an interface module adapted to interface with a memory and to retrieve a digital representation of the first time-reverse derived ultrasonic signal stored in the memory.

Optionally, the time-reverse derived ultrasonic signal is based on a 1-bit signal.

Optionally, the transducer unit is adapted to emit a second time-reverse derived ultrasonic signal which temporally overlaps the first time-reverse derived ultrasonic signal; the second time-reverse derived ultrasonic signal is focused on the focal point; and the second time-reverse derived ultrasonic signal has a second frequency characteristic which is different than the first frequency characteristic of the first time-reverse derived ultrasonic signal.

Optionally, the device further comprises a second transducer unit adapted to emit a second time-reverse derived ultrasonic signal which temporally overlaps the first time-reverse derived ultrasonic signal, wherein the second time-reverse derived ultrasonic signal is focused on the focal point, and wherein the second time-reverse derived ultrasonic signal has a second frequency characteristic which is different than the first frequency characteristic of the first time-reverse derived ultrasonic signal.

There is frrther provided, in accordance with an embodiment of the disclosure, a non-volatile memory device adapted to be read by a soft tissue destruction device, comprising a first time-reverse derived ultrasonic signal having a first frequency characteristic; and a datum pertaining to a relative location of a transmitter and a receiver, wherein the datum corresponds to said first time-reverse derived ultrasonic signal.

Optionally, said first time-reverse derived ultrasonic signal is a 1-bit signal.

Optionally, the memory device further comprises a second time-reverse derived ultrasonic signal having a second frequency characteristic which is different than said first frequency characteristic of said first time-reverse derived ultrasonic signal.

There is fuirther provided, in accordance with an embodiment of the disclosure, a user interface adapted to control a soft tissue destruction device, the user interface comprising a user-selectable ultrasonic focus parameter pertaining to a relative position of a transducer unit and a focal point within the soft tissue.

Optionally, the user interface further comprises a second ultrasonic focus parameter pertaining to a spatial coverage of at least one time-reverse derived ultrasonic signal adapted to be emitted from the soft tissue destruction device.

Optionally, the user interface further comprises a second ultrasonic focus parameter pertaining to at least one frequency value of a time-reverse derived ultrasonic signal adapted to be emitted from the soft tissue destruction device.

Optionally, the user interface further comprises a second ultrasonic focus parameter pertaining to at least two frequency values of corresponding at least two time-reverse derived ultrasonic signals adapted to be emitted from the soft tissue destruction device.

Optionally, the user interface further comprises a second ultrasonic focus parameter pertaining to a voltage amplitude adapted to excite a transducer of the soft tissue destruction device.

Optionally, the user interface further comprises a second ultrasonic focus parameter pertaining to a power level adapted to excite a transducer of the soft tissue destruction device.

There is further provided, in accordance with an embodiment of the disclosure, a method for producing a time reversed signal for destroying a fat tissue, the method comprising emitting an electrical signal towards a transducer unit being essentially in contact with a tissue simulating medium; receiving, using a sensor submerged within the tissue simulating medium, an ultrasonic signal derived from at least the electrical signal; converting the ultrasonic signal to a digital signal; and time-reversing the digital signal to produce a time-reversed signal.

Optionally, the transducer unit comprises at least one transducer attached to at least one resonator.

Optionally, the method further comprises storing the time-reversed signal in a memory, along with a corresponding datum pertaining to a relative location of the transducer unit and the sensor.

There is further provided, in accordance with an embodiment of the disclosure, a method for destroying a fat tissue, comprising retrieving a time-reversed digital signal and a datum containing a corresponding relative location of a transducer unit and a sensor from a memory; converting the time-reversed signal to an electrical time-reversed signal; and emitting the electrical time-reversed signal towards the transducer unit, so that a time-reversed ultrasonic signal created by the transducer unit is focused substantially at a center point of the target treatment area and destroys at least some of the fat tissue contained within the target treatment area.

There is further provided, in accordance with an embodiment of the disclosure, a method for creating a time-reverse derived signal adapted to destroy soft tissues, comprising time-reversing a signal emitted from a transmitter and received by a receiver.

Optionally, the transmitter is a transducer unit and the receiver is a sensor embedded in a tissue simulating medium.

Optionally, the transmitter is a sensor embedded in a tissue simulating medium and the receiver is a transducer unit.

In some embodiments, the soft tissues are adipose tissues.

In some embodiments, the method further comprises storing the time-reverse derived signal in a memory, along with a corresponding datum pertaining to a relative location of the transducer unit and the sensor.

In some embodiments, the method further comprises creating a second time-reverse derived signal by time-reversing a second signal, wherein the second signal is emitted from a transducer associated with the transducer unit, and wherein the second signal is received by the sensor, and wherein the second signal has a frequency which is different than a frequency of the signal.

In some embodiments, the method further comprises creating a second time-reverse derived signal by time-reversing a second signal, wherein the second signal is emitted from the sensor, and wherein the second signal is received by a transducer associated with the transducer unit, and wherein the second signal has a frequency which is different than a frequency of the signal.

In some embodiments, the method further comprises converting the signal to a 1-bit signal.

In some embodiments, the method further comprises converting the time-reverse derived signal to a 1-bit signal.

In some embodiments, the method further comprises converting the second signal to a 1-bit signal.

In some embodiments, the method further comprises converting the second time-reverse derived signal to a 1-bit signal.

There is further provided, in accordance with an embodiment of the disclosure, a method for destroying a soft tissue within a focal area, comprising emitting a time-reverse derived ultrasonic signal focused on a focal point within the focal area.

Optionally, the soft tissue is an adipose tissue.

Optionally, the time-reverse derived ultrasonic signal corresponds to a signal received by a sensor embedded in a tissue simulating medium.

Optionally, the time-reverse derived ultrasonic signal corresponds to a signal received by a transducer.

Optionally, the time-reverse derived ultrasonic signal induces cavitation within the focal area.

Optionally, the time-reverse derived ultrasonic signal is based on a 1-bit signal.

Optionally, the method further comprises emitting a second time-reverse derived ultrasonic signal which temporally overlaps the time-reverse derived ultrasonic signal, wherein the second time-reverse derived ultrasonic signal is focused on the focal point. The second time-reverse derived ultrasonic signal optionally has a frequency which is different than a frequency of the time-reverse derived ultrasonic signal. The second time-reverse derived ultrasonic signal is optionally emitted from a second transducer unit, which is not a transducer unit from. which the time-reverse derived ultrasonic signal is emitted.

Optionally, the method further comprises emitting a second time-reverse derived ultrasonic signal, which temporally overlaps the time-reverse derived ultrasonic signal, wherein the second time-reverse derived ultrasonic signal is focused on a second focal point. The second time-reverse derived ultrasonic signal optionally has a frequency, which is different than a frequency of the time-reverse derived ultrasonic signal. The second time-reverse derived ultrasonic signal is optionally emitted from a second transducer unit, which is not a transducer unit from which the time-reverse derived ultrasonic signal is emitted.

There is further provided, in accordance with an embodiment of the disclosure, a system for creating a time-reverse derived signal adapted to destroy a soft tissue, comprising a transducer unit adapted to emit an ultrasonic signal towards a tissue simulating medium; a sensor adapted to be embedded in the tissue simulating medium and adapted to receive the ultrasonic signal and convert it into an electrical signal; an analog-to-digital converter adapted to convert the electrical signal to a digital signal; and a signal processor adapted to time-reverse the digital signal and produce a time-reverse derived signal.

Optionally, the soft tissue is an adipose tissue.

Optionally, the system further comprises a memory adapted to store the time-reverse derived signal, along with a corresponding datum pertaining to a relative location of the transducer unit and the sensor.

Optionally, the transducer unit is adapted to emit a second ultrasonic signal towards the tissue simulating medium, wherein the second ultrasonic signal has a frequency which is different than a frequency of the ultrasonic signal.

Optionally, the system further comprises a second transducer unit adapted to emit a second ultrasonic signal towards the tissue simulating medium, wherein the second ultrasonic signal has a frequency which is different than a frequency of the ultrasonic signal.

Optionally, the time-reverse derived signal is a 1-bit signal.

There is further provided, in accordance with an embodiment of the disclosure, a device adapted to destroy a soft tissue within a focal area, comprising a transducer unit adapted to emit a time-reverse derived ultrasonic signal focused on a focal point within the focal area.

Optionally, the soft tissues are adipose tissues.

Optionally, the time-reverse derived ultrasonic signal corresponds to a signal received by a sensor embedded in a tissue simulating medium.

Optionally, the time-reverse derived ultrasonic signal corresponds to a signal received by a transducer.

Optionally, the time-reverse derived ultrasonic signal is adapted to induce cavitation within the focal area.

Optionally, the device further comprises a memory adapted to store a digital representation of at least the time-reverse derived ultrasonic signal.

Optionally, the device further comprises an interface module adapted to interface with a memory and to retrieve a digital representation of at least the time-reverse derived ultrasonic signal stored in the memory.

Optionally, the transducer unit is adapted to emit a second time-reverse derived ultrasonic signal, which temporally overlaps the time-reverse derived ultrasonic signal; the second time-reverse derived ultrasonic signal is focused on the focal point; and the second time-reverse derived ultrasonic signal has a frequency which is different than a frequency of the time-reverse derived ultrasonic signal.

Optionally, the device further comprises a second transducer unit adapted to emit a second time-reverse derived ultrasonic signal, which temporally overlaps the time-reverse derived ultrasonic signal, wherein the second time-reverse derived ultrasonic signal is focused on the focal point, and wherein the second time-reverse derived ultrasonic signal has a frequency which is different than a frequency of the time-reverse derived ultrasonic signal.

In some embodiments, the second time-reverse derived ultrasonic signal is based on a 1-bit signal.

In some embodiments, the second time-reverse derived ultrasonic signal is based on a 1-bit signal.

There is further provided, in accordance with an embodiment of the disclosure, a memory device adapted to be read by a soft tissue destruction device, comprising at least one time-reverse derived signal.

Optionally, the memory is non-volatile.

Optionally, the time-reverse derived signal is a 1-bit signal.

Optionally, the at least one time-reverse derived signal comprises at least two time-reverse derived signals. The at least two time-reverse derived signals optionally have different frequency characteristics.

There is further provided, in accordance with an embodiment of the disclosure, a user interface adapted to control a soft tissue destroying device, comprising at least one user-selectable ultrasonic focus parameter.

Optionally, the ultrasonic focus parameter is a relative position of a transducer unit and a focal point within the soft tissue.

Optionally, the ultrasonic focus parameter is a spatial coverage of at least one time-reverse derived ultrasonic signal emitted from the soft tissue destroying device.

Optionally, the ultrasonic focus parameter is at least one frequency value of a time-reverse derived ultrasonic signal emitted from the soft tissue destroying device.

Optionally, the ultrasonic focus parameter is at least two frequency values of corresponding at least two time-reverse derived ultrasonic signals emitted from the soft tissue destroying device.

Optionally, the ultrasonic focus parameter is a voltage amplitude used to excite a transducer of the soft tissue destroying device.

Optionally, the ultrasonic focus parameter is a power level used to excite a transducer of the soft tissue destroying device.

There is further provided, in accordance with an embodiment of the disclosure, a method for creating two or more time-reverse derived signals adapted to destroy a soft tissue, comprising time-reversing two or more signals having different frequency characteristics; wherein the two or more signals are emitted from at least one transducer unit and received by a sensor embedded in a tissue simulating medium.

Optionally, the soft tissue is an adipose tissue.

Optionally, the method further comprises storing the two or more time-reverse derived signals in a memory, along with a corresponding datum pertaining to a relative location of the at least one transducer unit and the sensor.

Optionally, the method further comprises converting at least one of the two or more signals to a 1-bit signal(s).

Optionally, the method fher comprises converting at least one of the two or more time-reverse derived signals to a 1-bit signal(s).

There is further provided, in accordance with an embodiment of the disclosure, a method for destroying a soft tissue within a focal area, comprising emitting at least two time-reverse derived ultrasonic signals having different frequency characteristics; wherein the at least two time-reverse derived ultrasonic signals are focused on a focal point within the focal area.

Optionally, the soft tissue is an adipose tissue.

Optionally, the at least two time-reverse derived ultrasonic signals correspond to at least two signals received by a sensor embedded in a tissue simulating medium.

Optionally, the at least two time-reverse derived ultrasonic signals correspond to at least two signals received by a transducer.

Optionally, the at least two time-reverse derived ultrasonic signals are adapted to induce cavitation within the focal area.

Optionally, one or more of the at least two time-reverse derived ultrasonic signals is based on a 1-bit signal(s).

There is fuirther provided, in accordance with an embodiment of the disclosure, a system for creating two or more time-reverse derived signals adapted to destroy a soft tissue, comprising at least one transducer unit adapted to emit two or more ultrasonic signals towards a tissue simulating medium, wherein the two or more ultrasonic signals have different frequency characteristics; a sensor embedded in the tissue simulating medium and adapted to receive the two or more ultrasonic signals and convert them to two or more electrical signals, respectively; an analog-to-digital converter adapted to convert the two or more electrical signals to two or more digital signals, respectively; and a signal processor adapted to time-reverse the two or more digital signals and produce two or more time-reverse derived signals, respectively.

Optionally, the soft tissue is an adipose tissue.

Optionally, the system further comprises a memory adapted to store the two or more time-reverse derived signals, along with a corresponding datum pertaining to a relative location of each of the at least one transducer unit and the sensor.

Optionally, at least one of the two or more time-reverse derived signals is a 1-bit signal.

There is further provided, in accordance with an embodiment of the disclosure, a device for destroying a soft tissue within a focal area, comprising at least one transducer unit adapted to emit two or more time-reverse derived ultrasonic signals having different frequency characteristics; wherein the two or more time-reverse derived ultrasonic signals are focused on a focal point within the focal area.

Optionally, the soft tissue is an adipose tissue.

Optionally, the two or more time-reverse derived ultrasonic signals correspond to two or more signals received by a sensor embedded in a tissue simulating medium.

Optionally, the two or more time-reverse derived ultrasonic signals correspond to two or more signals received by a transducer.

Optionally, the two or more time-reverse derived ultrasonic signals are adapted to induce cavitation within the focal area.

Optionally, the device further comprises a memory adapted to store two or more digital representations of the two or more time-reverse derived ultrasonic signals, respectively.

Optionally, the device further comprises an interface module adapted to interface with a memory and to retrieve two or more digital representations of the two or more time-reverse derived ultrasonic signals, respectively, stored in the memory.

Optionally, one or more of the at least two time-reverse derived ultrasonic signals is based on a 1-bit signal.

### BRIEF DESCRIPTION OF THE FIGURES

Examples illustrative of embodiments of the disclosure are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures are listed below.

FIG. 1 schematically shows a forward calibration configuration;

FIG. 2 schematically shows a graphic representation of a pulse;

FIGS. 3A-3K schematically show transducer unit configurations;

FIG. 4 schematically shows a tissue simulating medium;

FIG. 5 schematically shows a block diagram of a signal processor;

FIG. 6 schematically shows graphic representations of ultrasonic signals;

FIG. 7 schematically shows graphic representations of time-reversed ultrasonic signals;

FIG. 8 schematically shows a flow chart of a forward calibration;

FIG. 9 schematically shows a graphic representation of an ultrasonic signal;

FIG. 10 schematically shows a graphic representation of a time-reversed ultrasonic signal;

FIG. 11 schematically shows a graphic representation of a signal and its 1-bit equivalent;

FIG. 12 schematically shows a backward calibration configuration;

FIG. 13 schematically shows a block diagram of a signal processor;

FIG. 14 schematically shows a flow chart of a backward calibration;

FIG. 15 schematically shows a Graphical User Interface (GUI);

FIG. 16 schematically shows a cross section view of treatment using a HIFU system;

FIG. 17 schematically shows a cross section view of treatment using a HIFU system;

FIG. 18 schematically shows a treatment using a HIFU system; and

FIG. 19 schematically shows a block diagram of a HIFU system.

### DETAILED DESCRIPTION

An aspect of some embodiments relates to a high-intensity focused ultrasound 30 (HIFU) system adapted to destroy soft tissues (the destroying hereinafter referred to as “histotripsy”), such as adipose tissues.

The following detailed description discloses methods, devices and systems adapted to calibrate such a HIFU system, so that it may emit an ultrasonic signal focused essentially on the soft tissues. In addition, the detailed description discloses a user interface, optionally a Graphical User Interface (GUI), adapted to allow selection of focus-related parameter(s) prior to and/or during a treatment using the HIFU system. Furthermore, methods, devices and systems enabling operation and usage of a calibrated HIFU system are also disclosed.

### HIFU System Calibration

In an embodiment, optionally prior to treatment, a HIFU system is calibrated using an acoustic time-reversal method, so that it may emit an ultrasonic signal focused essentially on soft tissues whose destruction is desired.

Forward Calibration Configuration

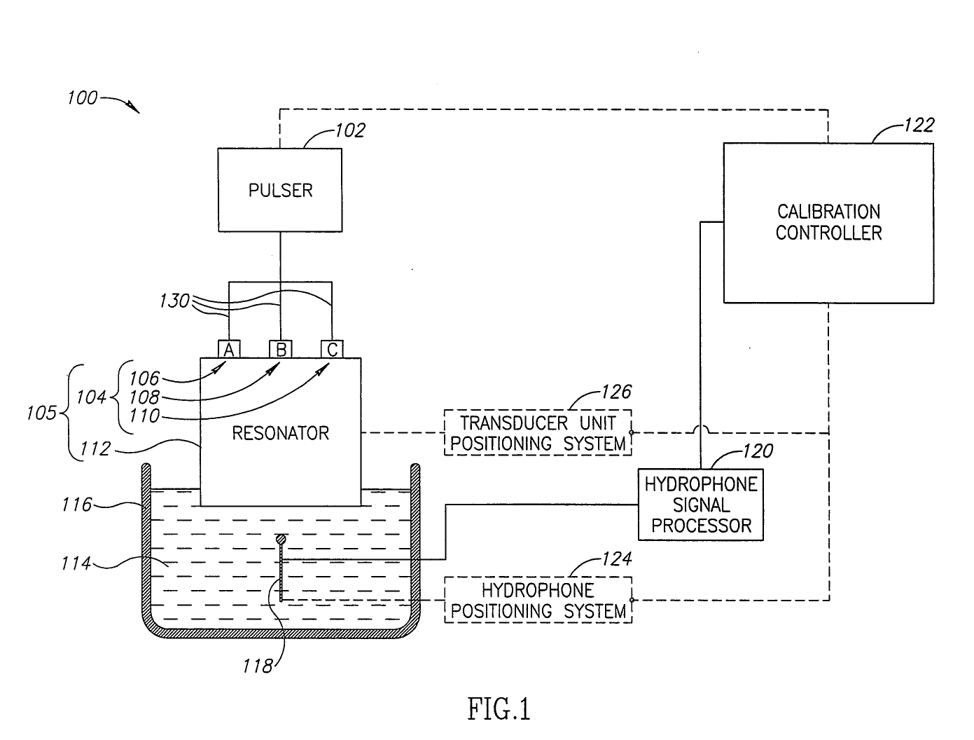

Reference is now made to FIG. 1, which shows an exemplary forward calibration configuration 100. Forward calibration configuration 100 may include a pulser 102, a transducer set 104 and a resonator 112 (hereinafter jointly referred to as transducer unit 105), a tissue simulating medium 114 optionally contained within a tank 116, a hydrophone 118, a hydrophone signal processor 120 and/or a calibration controller 122. Forward calibration configuration 100 optionally includes a hydrophone positioning system 124 and/or a transducer unit positioning system 126.

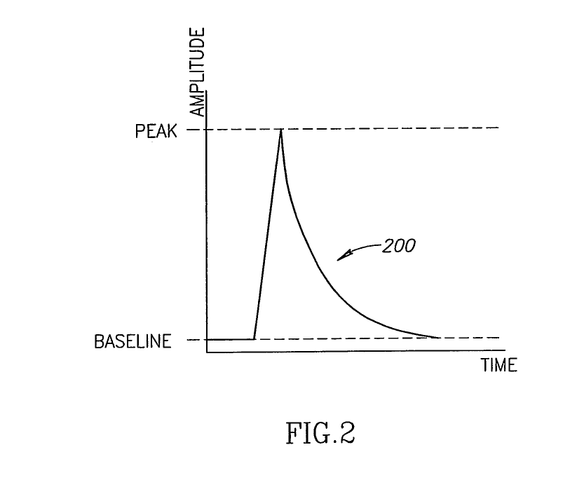

Pulser 102 is a device adapted to emit an electrical pulse, which may essentially be a rapid increase in an amplitude of a signal from a baseline value to a higher value, followed by a return to approximately the baseline value. FIG. 2 schematically shows an exemplary graphic demonstration of a pulse amplitude as a function of time. A graph 200 shows a signal which rapidly increases in amplitude from a baseline level to a peak level, and then declines to approximately the baseline level. Other pulses (not shown) may exhibit a differently shaped increase in amplitude and/or a differently shaped decline. Additionally or alternatively, other pulses may exhibit a return to a level higher or lower than the baseline level.

Referring now to FIG. 1, Pulser 102 may be adapted to selectively emit a pulse towards one or more transducers of a set, such as transducer set 104, over N number of channels 130. The selective emitting is optionally performed using an N-channel pulsing mechanism (not shown) which is essentially connected to or integrally formed with pulser 102. The N-channel pulsing mechanism may be controlled by calibration controller 122. Optionally, pulser 102 is accompanied by one or more additional pulsers (not shown), each adapted to selectively emit a pulse towards one or more transducers of one or more transducer sets.

Transducer set 104 may include one or more ultrasonic transducers, orderly arrayed and/or randomly/arbitrarily arranged. Usage of multiple transducers may enable emitting ultrasonic signals having different and/or same frequencies, amplitudes, focuses and/or the like. Such emitting may be performed simultaneously, consecutively or in a combination thereof. For simplicity of presentation, forward calibration configuration 100 is shown with three transducers, transducer A 106, transducer B 108 and transducer C 110. However, persons of skill in the art will recognize that a different number of transducers may be used for achieving different combinations of frequencies, amplitudes, focuses and/or the like. As one example, multiple transducers with some having different resonant frequencies may be used. Generally, usage of a plurality of transducers may increase a signal-to-noise ratio—the ratio between a “good”, desired signal and a “bad”, undesired noise and interference.

Transducers A 106, B 108 and C 110 of transducer set 104 are optionally piezoelectric transducers made of piezoelectric crystals, ceramics and/or other piezoelectric materials, adapted to convert electrical energy into ultrasound. When transducers A 106, B 108 and C 110 are electrified, vibrations (sometimes referred to as “oscillations”) are excited within their body, producing ultrasonic waves.

Transducers A 106, B 108 and C 110 of transducer set 104 are optionally attached to resonator 112, by such means as using glue and/or other chemical and/or mechanical means of attachment. Optionally, transducers A 106, B 108 and C 110 are attached to an intermediary layer (not shown) which is, in turn, attached to resonator 112. The intermediary layer may be made of materials such as metal, polymer, fiberglass and/or the like.

Resonator 112 is a device exhibiting acoustic resonance behavior—it oscillates at some frequencies with greater amplitude than at others. Generally, resonator 112 may be defined as a wave guide—a physical structure that guides ultrasound waves in a desired pattern, direction and/or the like. Resonator 112 may be made of aluminum and/or of other solids or liquids having desired resonation characteristics.

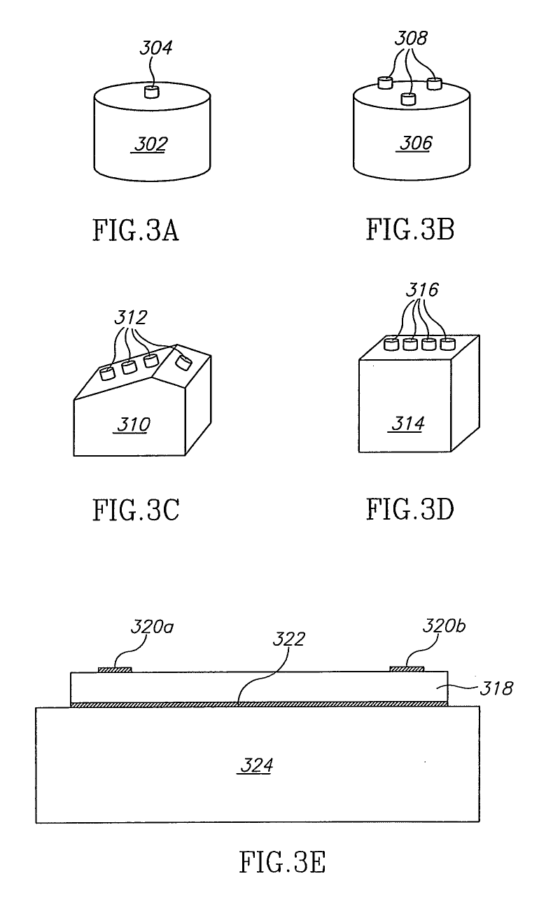

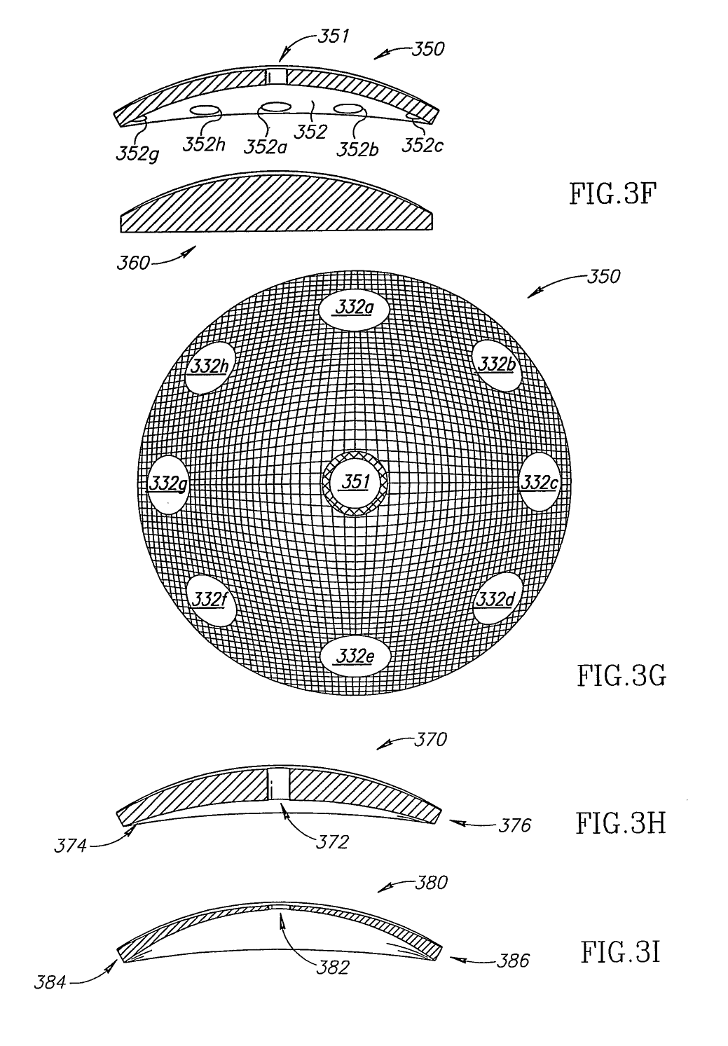

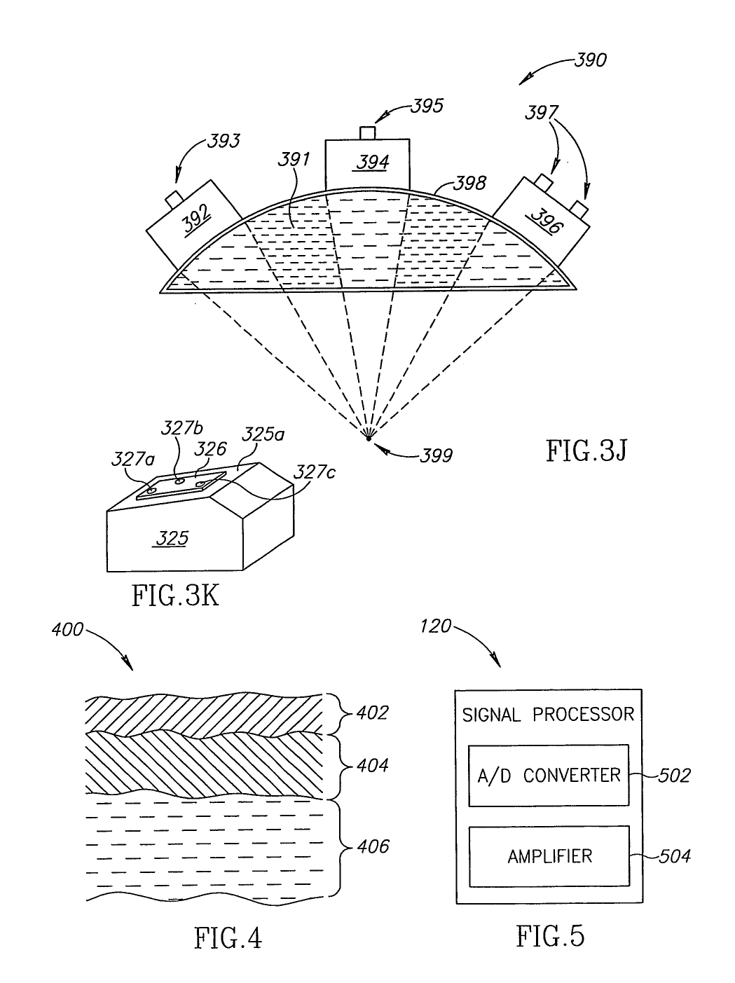

Reference is now made to FIGS. 3A-3K, which show exemplary transducer unit configurations, of many possible options.

A resonator 302 is an essentially solid cylinder. A single transducer 304 is present on a top facet of resonator 302.

A resonator 306 is also an essentially solid cylinder, having, for example, three transducers 308 on its top facet.

A resonator 310 is an essentially solid pentagon-shaped device having, for example, four transducers 312 laid out on its two top facets. Having a resonator with a complex top surface shape may, in some scenarios, enhance its functionality.

A resonator 314 is an essentially solid cube, having, for example, four transducers 316 on its top facet.

A piezo plate 318 is made of a piezoelectric material, and is either flat, concave, convex or otherwise shaped. For simplicity of presentation, piezo plate 318 is shown flat. One or more resonators may be attached to piezo plate 318, for example a resonator 324. Piezo plate 318 may have one or more electrodes on each of its top and bottom surfaces, such as electrodes 320a and/or 320b located on its top surface, and an electrode 322 located on its bottom surface. An electrode may be an essentially thin layer of metal, which may essentially be grounded or connected to a power source adapted to deliver electrical current to the electrode (to “electrify” it). When an electrode coupled to a piezoelectric body (such as piezo plate 318) is electrified while another electrode is essentially grounded, vibrations are excited in the piezoelectric body, and are usually limited to a region of the piezoelectric body that is in close proximity and between the electrodes. This region may be referred to as a transducer. If a piezoelectric body, such as piezo plate 318, has multiple distinct electrodes, such as electrodes 320a and/or 320b, then each region in close proximity to each of these electrodes may be regarded as a separate transducer.

Electrifying electrodes of piezo plate 318 may lead to an excitation of resonations (or “reflections”) within resonator 324, which is positioned adjacent to the piezo plate, and optionally attached to it using a glue and/or mechanical means.

Optionally, some or all of electrodes 320a-b and/or 322 are positioned and/or shaped in such a way that there is essentially no “cross-talk” between piezoelectric regions near each of these electrodes. Additionally or alternatively, piezo plate 318 is made of such a piezoelectric material that it does not essentially exhibit the “cross-talk” phenomenon. In other words, electrification of two electrodes does not essentially trigger vibrations in piezoelectric regions not associated with these electrodes.

Somewhat similar to piezo plate 318, a concave piezo plate 350 (FIG. 3F) is made of a piezoelectric material, and optionally has an aperture 351 in its center area. Aperture 351 may encompass one or more of a variety of auxiliary appliances, such as, for example, a contact sensor adapted to sense contact of a transducer unit with a treated area. Concave piezo plate 350 may have a uniform thickness along its area. Alternatively, concave piezo plate 350 may have a non-uniform thickness. For example, it may be relatively thicker or thinner in specific one or more areas, such as areas essentially surrounding electrodes like electrodes 352a-h. Concave piezo plate 350 may include one or more electrodes located essentially on its bottom surface 352, such as electrodes 352a-h. Referring now to FIG. 3G which shows a top view of concave piezo plate 350, the concave piezo plate may include electrodes on its top surface, such as electrodes 332a-h, that correspond to electrodes 352a-h located on the opposite side. FIG. 3G shows eight round electrodes, but persons of skill in the art will recognize that the concept of placing electrodes on the top and bottom surfaces of concave piezo plate 350 may include any other number of electrodes having any other shape, size and/or location. Optionally, bottom surface 352 of concave piezo plate 350 includes a single electrode which spreads over a substantial portion of the bottom surface, up to the entirety of the bottom surface. Alternatively, bottom surface 352 may include a lower or a greater number of electrodes than electrodes located on the top surface of concave piezo plate 350.

A resonator 360 may be attached to bottom surface 352 of concave piezo plate 350. Resonator 360 is optionally a device having a convex top surface which may correspond to a concavity of a bottom surface 352 of concave piezo plate 350. When resonator 360 is fitted within the concavity of bottom surface 352, essentially its entire convex top surface may contact bottom surface 352.

A plate 370 and a plate 380 are optional variations of concave piezo plate 350. Plate 370 is shown having a thicker body around an area of an aperture 372 and a thinner body around its circumferential area, such as near areas 374 and 376. Plate 380 is shown having a thinner body around an area of an aperture 382 and a thicker body around its circumferential area, such as near areas 384 and 386.

FIG. 3J shows an exemplary configuration 390 including a plurality of resonators, such as a resonator 392, a resonator 394 and a resonator 396, arranged on a convex platform 398. For simplicity of presentation, FIG. 3J shows three resonators, but any number of resonators may be arranged on convex platform 398. Convex platform 398 is optionally a hollow hemispherical device which may contain a fluid 391. Other convex platforms (not shown) may be merely convex plates. Resonators 392, 394 and 396 may each be accompanied by one or more transducers on its top surface. For example, resonator 392 may have one transducer 393, resonator 394 may have one transducer 395, and resonator 396 may have two transducers 397. The arched arrangement of resonators 392, 394 and 396 on convex platform 398 may enable focusing ultrasonic signals emitted by at least one of these resonators on a focal point such as a focal point 399.

FIG. 3K shows an essentially pentagon-shaped resonator 325 having a piezo plate 326 on its top surface 325a. Piezo plate 326 may be similar to or different than piezo plate 324 of FIG. 3E. Piezo plate 326 may have one or more electrodes on its top surface, such as electrodes 327a, 327b and/or 327c, as well as one or more electrodes on its bottom surface (not shown).

Persons of skill in the art will recognize that these examples, which pertain to shapes and quantities of transducers, resonators, electrodes and/or other related elements, are not exhaustive and are meant for demonstrative purposes only. Transducers, resonators, electrodes and/or other related elements of different quantities and shapes are explicitly intended to be within the scope of this disclosure.

Referring now to FIG. 1, optional transducer unit positioning system 126 may control three-dimensional positioning of transducer unit 105.

Tissue simulating medium 114 is a liquid, a solid or a combined liquid-solid medium, having acoustic impedance characteristics similar to those of soft tissue such as skin and/or fat tissue. Tissue simulating medium 114 may be, for example, water, contained within tank 116. Experiments show that the acoustic impedance of water is similar to that of soft tissue such as fat. Tissue simulating medium 114 may also be, for example, oil, contained within tank 116. Additional experiments show that the acoustic impedance of oil is also similar to that of soft tissue such fat.

Tissue simulating medium 114 may optionally be formed with layers, to simulate various layers of skin, fat and muscles. Reference is now made to FIG. 4, which shows an exemplary layered tissue simulating medium 400, which includes three layers: Epidermis simulator 402, dermis simulator 404 and subcutis simulator 406. Each of these layers is optionally formed having acoustic impedance similar or identical to that of the corresponding skin layer. Epidermis simulator 402 and dermis simulator 404 are optionally substantially thinner than subcutis simulator 406, which simulates a layer mainly containing fat tissue. Optionally, instead of having separate epidermis simulator 402 and dermis simulator 404 layers, a single layer (not shown) simulating essentially both may be formed.

Referring now to FIG. 1, hydrophone 118 is embedded within tissue simulating medium 114, optionally within a layer simulating fat tissue such as subcutis simulator 406 of FIG. 4. Hydrophone 118, which may also be referred to as a “sensor”, is an ultrasound sensor, optionally made of a piezoelectric material. Hydrophone 118 may be adapted to receive an ultrasonic signal (such as a signal which travels through tissue simulating medium 114) and convert it to an electrical signal. Additionally or alternatively, hydrophone 118 may be adapted to be pulsed with an electrical signal and to emit a corresponding ultrasonic signal.

Optional hydrophone positioning system 124 may control three-dimensional positioning of hydrophone 118.

Hydrophone signal processor 120 may receive an electronic signal from hydrophone 118, the signal representing ultrasonic waves sensed by the hydrophone. FIG. 5 shows a block diagram of hydrophone signal processor 120, which may include an analog/digital (A/D) converter 502 and an amplifier 504. A/D converter 502 is an electronic device adapted to convert electronic signals received from hydrophone 118 (FIG. 1) to digital data, so that the digital data represents ultrasonic waves sensed by the hydrophone. Amplifier 504 is an electronic device adapted to amplify electronic signals received from hydrophone 118, so that these signal are more easily identified and treated.

Referring now to FIG. 1, calibration controller 122 is a computerized device optionally adapted to control an operation of one of more components of forward calibration configuration 100 and to optionally store calibration-related information. For example, calibration controller 122 may control a positioning of transducer unit 105 and/or hydrophone 118, essentially using transducer unit positioning system 126 and/or hydrophone positioning system 124, respectively. As another example, calibration controller 122 may control one or more electrical pulse(s) emitted by pulser 102 towards some or all transducers of transducer set 104. Yet another example is a control of hydrophone signal processor 120 and its signal amplification and/or A/D conversion operation(s).

Aspects of Time-Reversal

An exemplary theoretic model for calibrating a HIFU system using a time reversal (TR) method may be structured as follows:

The first step of the TR procedure is to receive the linear system impulse response as h(t). It is supposed to have the structure: h(t)=H(t)\tilde{h}(t). Here \tilde{h}(t) is the stationary Gaussian process with the autocorrelation function φ(t₁−t₂)= [symbol] \tilde{h}(t₁)\tilde{h}(t₂) [symbol] , φ(0)=1, the autocorrelation being a quadratically integrated function with the decay time τ_{c}. The envelope H(t) is believed to be a slowly decaying function (t_{H}⁻¹=−d ln H(t)/dt<<τ_{c}⁻¹).

On the second step, the impulse response is windowed by the rectangular window of duration T, then the inverted version of the response hᵢₙᵥₑᵣₜ(t)=Ah(T−t) is sentto input of the same linear system. The corresponding output is:

$$r ( t ) = A {\int }_{0}^{t} h ( τ ) {h}_{invert} ( t - τ ) d τ = A {\int }_{0}^{t} h ( τ ) h ( T - t + τ ) d τ \qquad ( 1.1 )$$

The number of important consequences follows from (1.1). First of all, the ensemble average of the output r(t) is proportional to the autocorrelation:

$$〈 r ( t ) 〉 = A ϕ ( T - t ) {\int }_{0}^{t} H ( τ ) H ( T - t + τ ) d τ \qquad ( 1.2 )$$

In particular,

$$〈 r ( T ) 〉 = AT {〈 {H}^{2} 〉}_{T} , {〈 {H}^{2} 〉}_{T} = \frac{1}{T} {\int }_{0}^{T} {H}^{2} ( τ ) d τ \qquad ( 1.3 )$$

The formulas (1.2), (1.3) present no data concerning the tolerance of the peak value r(T) and the noise level outside the peak region [T−τ_{c};T+τ_{c}]. The fourth moment analysis provides necessary estimations. Hereinafter we suppose that T>>τ_{c}. The following formulas are main asymptotic with regards to ratio τ_{c}/T. Considering the noise level outside the peak region but close enough to it (|t−T|≧τ_{c}), the signal-to-noise ratio is

$$SNR = \frac{〈 r ( T ) 〉}{\sqrt{〈 {r}^{2} ( t ) 〉}} {\vert}_{\vert t - T \vert \geq {τ}_{c}} = \sqrt{\frac{T}{{C}_{ϕ} {τ}_{c}}} \frac{{〈 {H}^{2} 〉}_{T}}{\sqrt{{〈 {H}^{4} 〉}_{T}}} , \\ {〈 {H}^{4} 〉}_{T} = \frac{1}{T} {\int }_{0}^{T} {H}^{4} ( τ ) d τ , {C}_{ϕ} = \frac{2}{{τ}_{c}} {\int }_{0}^{T} {ϕ}^{2} ( τ ) d τ \qquad ( 1.4 )$$

The relative tolerance of the peak value r(T) is

$$\frac{\sqrt{〈 {r}^{2} ( T ) 〉 - {〈 r ( T ) 〉}^{2}}}{〈 r ( T ) 〉} = \sqrt{\frac{2 {C}_{ϕ} {τ}_{c}}{T}} \frac{\sqrt{{〈 {H}^{4} 〉}_{T}}}{{〈 {H}^{2} 〉}_{T}} = \frac{\sqrt{2}}{SNR} \qquad ( 1.5 )$$

The constant C_{φ}=O(1)in (1.4), (1.5) depends on the actual form of the autocorrelation φ. In the case φ(τ)=exp(−τ/τ_{c}), for example, C_{φ}=1. It is seen from (1.4) and (1.5) that SNR grow as the impulse response duration T increases while the relative tolerance of the peak value r(T) decreases. In the simplest case of constant envelope H(t), they are proportional to \sqrt{N} and 1/\sqrt{N} respectively, where N=T/τ_{c}. It means that the peak value r(T) is the self-averaging value. The influence of the envelope on SNR can be clarified in the practically important case of exponential decay of the envelope: H(t)=H₀ exp(−t/t_{H}). It leads to the simple formulae:

$$ψ ( T / {t}_{H} ) = \frac{\sqrt{{〈 {H}^{4} 〉}_{T}}}{{〈 {H}^{2} 〉}_{T}} = \frac{\sqrt{1 - exp ( - 4 T / {t}_{H} )}}{1 - exp ( - 2 T / {t}_{H} )} \sqrt{\frac{T}{{t}_{H}}} \qquad ( 1.6 )$$

When T<0.5t_{H}, this function is close to 1 hence the envelope does not affect SNR estimation. Contrarily, if T>2t_{H} then ψ(T/t_{H})≈\sqrt{T/t_{H}}. In this case, the estimation of SNR becomes as follows:

$$SNR = \sqrt{\frac{{t}_{H}}{{C}_{ϕ} {τ}_{c}}} \qquad ( 1.4 A )$$

It does not depend on the duration T. Therefore, the practically important consequence is: as the duration of impulse response exceeds the characteristic time of its envelope decay, no meaningful improvement of SNR can be achieved. The number of additional consequences can be gained from the formula (1.4-1.6):
The more the difference between impedances of elastic resonator and acoustic load the better SNR is expected because of growth of t_{H}
SNR improves with a growth of the resonator height, that is, the length between interface with the acoustical load and the opposite surface
SNR is proportional to the square root of the central frequency of a transducer because τ_{c}˜ω⁻¹
SNR degrades with growth of the transducer's quality factor Q because it results in increase of τ_{c}
SNR is proportional to the square root of the number of radiators, the latter being spaced as to provide statistically independent impulse responses at a focal point, because the time reversed signals are summed constructively at the point t=T, while outside the interval [T−τ_{c};T+τ_{c}], their contributions are weakly correlated.

An exemplary numeric estimation may be structured as follows. The estimations (1.4-1.6) are the asymptotic ones; therefore, some kind of additional justification of those formulas is useful. The one bit quantization of the impulse response which is of common use in TR techniques is another and even more important reason for the numerical simulation of the regarded processes. The simulation can show whether or of the formulas (1.4-1.6) keep being valid after the quantization.

The stochastic model that is chosen for simulation is the sequence of two linear filters. The impulse response of the first filter is supposed to be a delta-correlated Gaussian process g(t) multiplied by slowly decaying exponent exp(−t/t_{H}), t_{H}>>ω₀⁻¹, where ω₀ is the cyclic eigen frequency of the harmonic oscillator with the quality Q, the oscillator being the second filter. The “white noise” g(t), [symbol] g(t₁)g(t₂) [symbol] =σ²δ(t₁−t₂), [symbol] g(t) [symbol] =0, is modeled by the uncorrelated Gaussian sequence gᵢ=g(iΔt), [symbol] gᵢgⱼ [symbol] =σ²δᵢⱼ/Δt. Here δᵢⱼ is Kronecker delta. The sampling rate is set as to provide ten intervals per the oscillator period. The resulting impulse response is approximately calculated as: h(tᵢ)=\tilde{h}(tᵢ)exp(−tᵢ/t_{H}), where the stationary Gaussian process \tilde{h}(tᵢ) is the convolution \tilde{h}(tᵢ)=[g\textcircled{×}osc](t₁) of the “white noise” g(t) with the response of the oscillator, the last one being: osc(t)=exp(−t/τ_{c})sin(ω₀t\sqrt{1−1/4Q²}), τ_{c}=2Q/ω₀. It is not difficult to show that for t₁, t₂>>τ_{c} the normalized autocorrelation of \tilde{h}(tᵢ) the normalized autocorrelation of \tilde{h}(tᵢ) approximately is:

$$\frac{〈 \tilde{h} ( {t}_{1} ) \tilde{h} ( {t}_{2} ) 〉}{〈 {\tilde{h}}^{2} ( t ≫ {τ}_{c} ) 〉} = ϕ ( {t}_{1} - {t}_{2} ) \approx exp ( - \vert {t}_{1} - {t}_{2} \vert / {τ}_{c} ) cos ( {ω}_{0} ( {t}_{1} - {t}_{2} ) \sqrt{1 - 1 / 4 {Q}^{2}} ) \qquad ( 1.7 )$$

The correspondent value of C_{φ}in (1.4) is approximately 0.5. The following simulation patterns have been obtained with the next setup:

| Central Frequency | Oscillator | Envelope Decay t_{H} | 1 bit Quantization |
|---|---|---|---|
| (MHz) | Quality Q | (μs) | Option |
| 1.0 | 4 | 1000 | Yes |

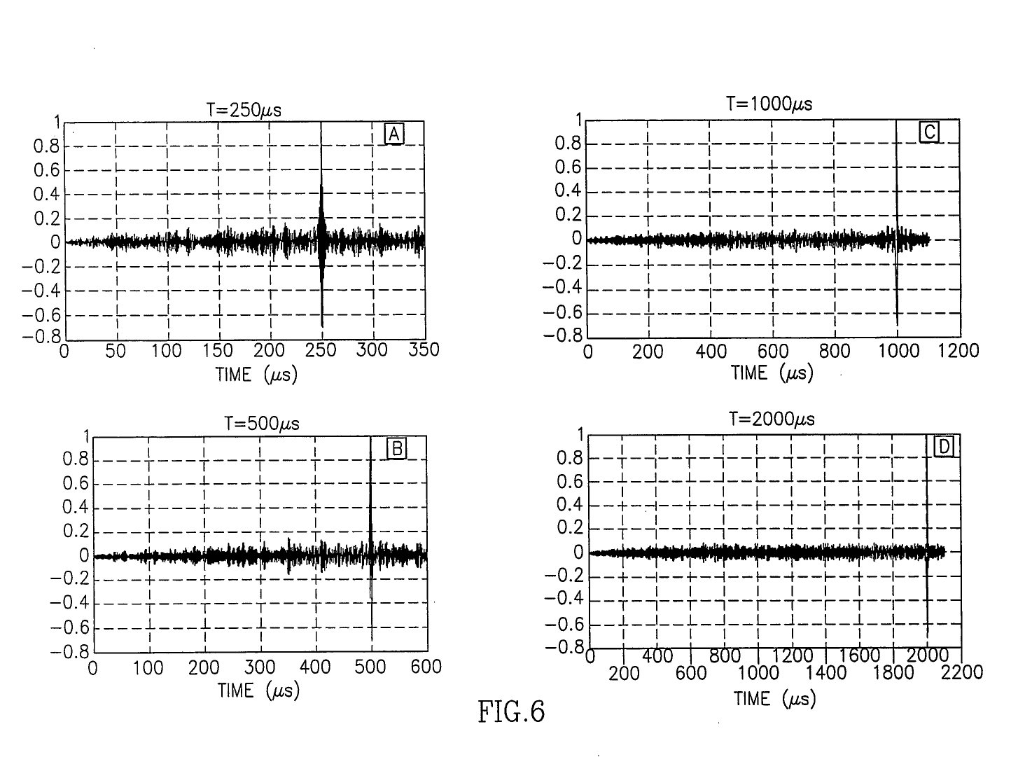

The different response durations T have been chosen as to demonstrate correspondence between predicted and experienced SNR in one random realization for the variety of relations between T and t_{H}. The noise level has been estimated by averaging of signal energy over the time period equal to 100_{τ}_{_{c}} and preceding peak zone. Because ergodicity of the noise seems to be a reasonable hypothesis, this averaging procedure is likely similar to the ensemble averaging. The next table summarizes SNR data, which correspond to signals presented in FIG. 6 (except for T=4000 μs).

| | | | | | |
|---|---|---|---|---|---|
| Response Duration T (μs) | 250 | 500 | 1000 | 2000 | 4000 |
| Predicted SNR | 19.6 | 27.0 | 34.6 | 38.9 | 39.6 |
| SNR | 20.6 | 23.3 | 37.8 | 39.6 | 41.4 |

The fairly close fit of the predicted SNR and the realized one is demonstrated by the table. The above-mentioned saturation effect as T≧2t_{H} is also clearly seen.

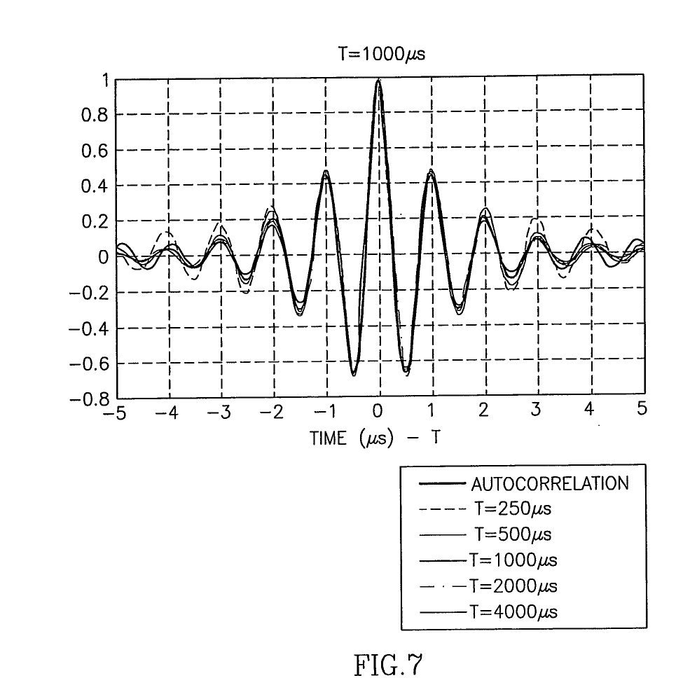

Shown in FIG. 7 are the TR signals around their peak positions. The autocorrelation (1.7) is also presented here for a comparison. This graph demonstrates both the validity of (1.2) and the above mentioned property of self-averaging TR signal at t=T.

Thus, the presented results of the numerical simulation confirm theoretical estimations (1.2-1.7) and show that those estimations are valid whether or not the one bit quantization of the impulse response is made.

A First Exemplary Calibration Method (Hereinafter “Forward Calibration”) In an embodiment, a forward calibration of a HIFU system is performed, essentially using calibration configuration 100, by emitting a calibration ultrasonic signal from a first location towards a tissue simulating medium, receiving the signal at a second location within the tissue simulating medium and deriving a time-reversed signal from the received signal. The time-reverse derived signal is stored in a memory, along with information pertaining to the relative position of the first and the second locations.

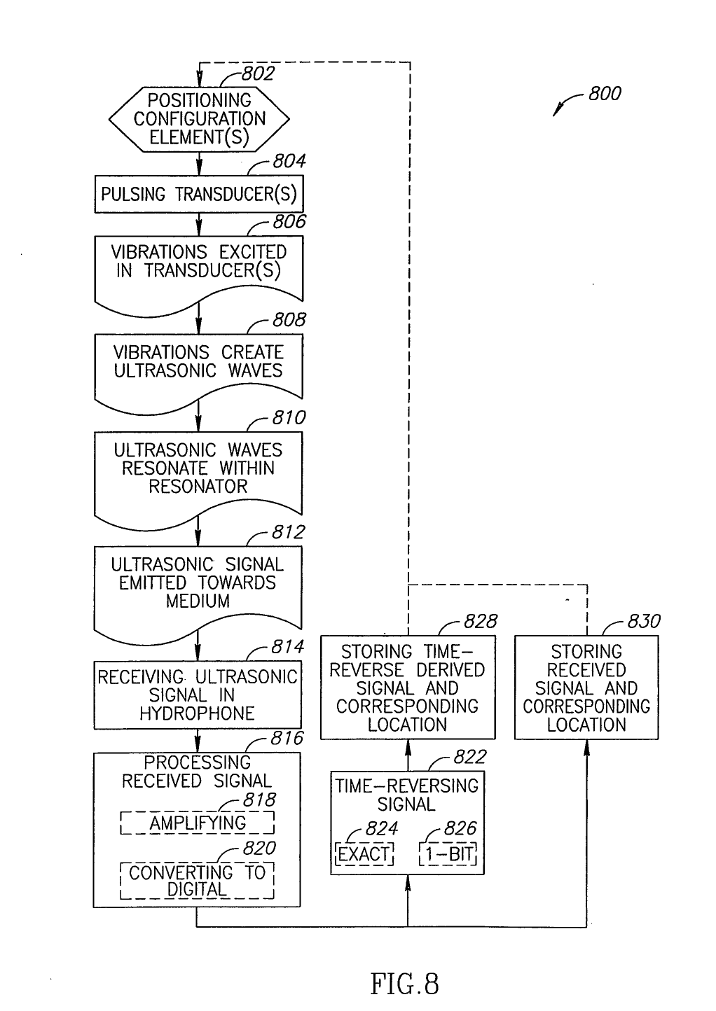

Reference is n ow made to FIG. 8, which shows a flow chart of a first exemplary calibration method (hereinafter “forward calibration”) 800. Description of forward calibration 800 is herein presented by referring to FIGS. 1 and 8 intermittently. Elements of FIG. 1 are notated by numerals ranging between 100-199, while elements of FIG. 8 are notated by numerals ranging between 800-899.

In a block 802, at least one element of forward calibration configuration 100 is positioned. For example, transducer unit positioning system 126 may be employed to position transducer unit 105. Yet another example is a positioning of hydrophone 118 using hydrophone positioning system 124. Calibration controller 122 optionally keeps track of the element positioning(s), so that a relative position of resonator 112 and/or transducer set 104 (hereinafter “first location”) and hydrophone 118 (hereinafter “second location”) is known. Optionally, the relative position is three-dimensional. Positioning of the at least one element(s) may be controlled by calibration controller 122, which optionally stores information pertaining to the relative position of the first and the second locations and/or stores the first and the second locations themselves.

In a block 804, pulser 102 emits an electrical pulse, also referred to as a “calibration pulse”, towards one or more transducer(s) of transducer set 104. Optionally, the signal is approximately 0.1 νs (microseconds) long.

In a block 806, the electrical pulse emitted by pulser 102 excites vibrations in the one or more pulsed transducer(s) of transducer set 104. In a block 808, the vibrations create ultrasonic waves.

In a block 810, the ultrasonic waves resonate and reflect within resonator 112 to which the one or more transducer(s) of transducer set 104 are essentially attached. The multiple reflections of the ultrasonic wave essentially produce an ultrasonic signal which is temporally longer than a signal that would have been produced by a standalone transducer or transducers.

In a block 812, the ultrasonic signal travels from resonator 112 and is essentially emitted towards tissue simulating medium 114.

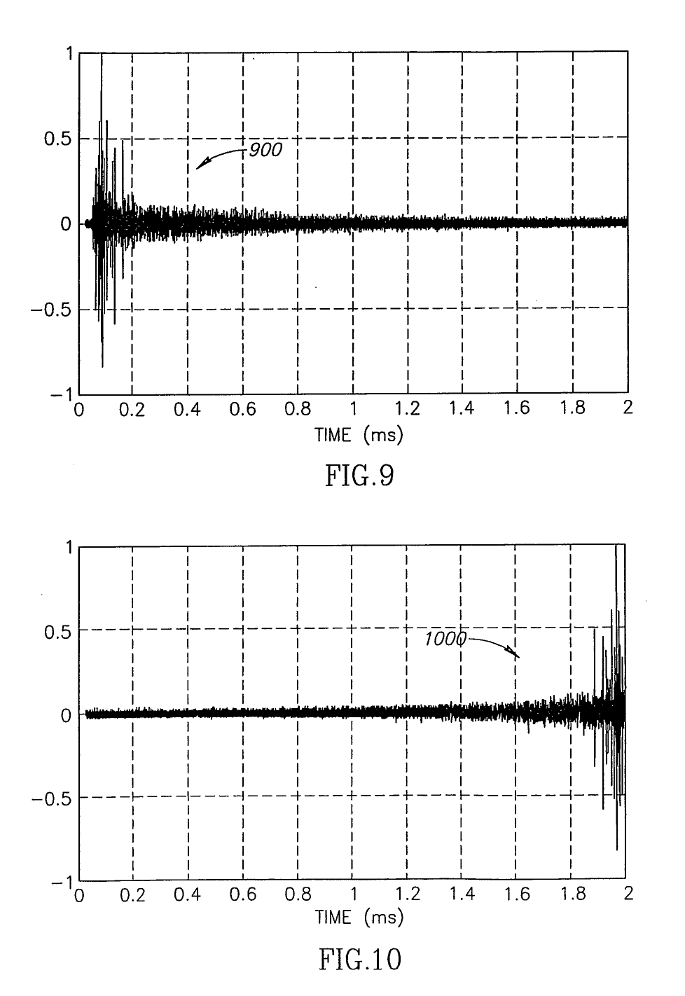

In a block 814, the ultrasonic signal is received by hydrophone 118. Optionally, hydrophone 118 receives the signal over a pre-defined time window, so that a latter portion of the signal may not be received and/or be omitted after receiving. Optionally, the pre-defined period of time is approximately 2000 μs (2 milliseconds). Limiting a length of the received signal may essentially enhance forward calibration 800, since resonant acoustic signals, in general, tend to become weak and/or less useful over time. FIG. 9 shows a graphic representation of an exemplary ultrasonic signal 900 received by hydrophone 118. As shown, signal 900 has several dominant, strong amplitudes in the range of 0-0.2 milliseconds, after which the signal slowly fades. In the range of 0.8 milliseconds and 2 milliseconds, for example, amplitude changes in the signal are minimal, compared to the strong amplitudes demonstrated earlier. As the signal fades, the fading portion may become less and less useful for forward calibration 800, and therefore, the time window may be pre-defined to omit this fading portion.

In a block 816, the signal may be processed. The signal is optionally amplified 818 using amplifier 504 (FIG. 5) and/or converted 820 to a digital signal using A/D converter 502 (FIG. 5). The amplification and/or the conversion may enable and/or support further manipulations to the signal. By way of example, converting the signal from analog to digital may allow its manipulation using a computer (such as calibration controller 122), which essentially handles digital data. Optionally, the processing of the hydrophone signal includes compensation for hydrophone-specific sensitivity imperfections. Each hydrophone, as it comes out of fabrication, has its own impulse response characteristic (in the time domain) or transfer function (in the frequency domain). Hydrophone manufacturers usually supply this data, which may be used in block 816 for compensating for these sensitivity imperfections.

Forward calibration 800 optionally splits, so that either a block 830 or a combination of blocks 822 and a block 828 is performed. Alternatively, both block 830 and the combination of blocks 822 and block 828 may be performed.

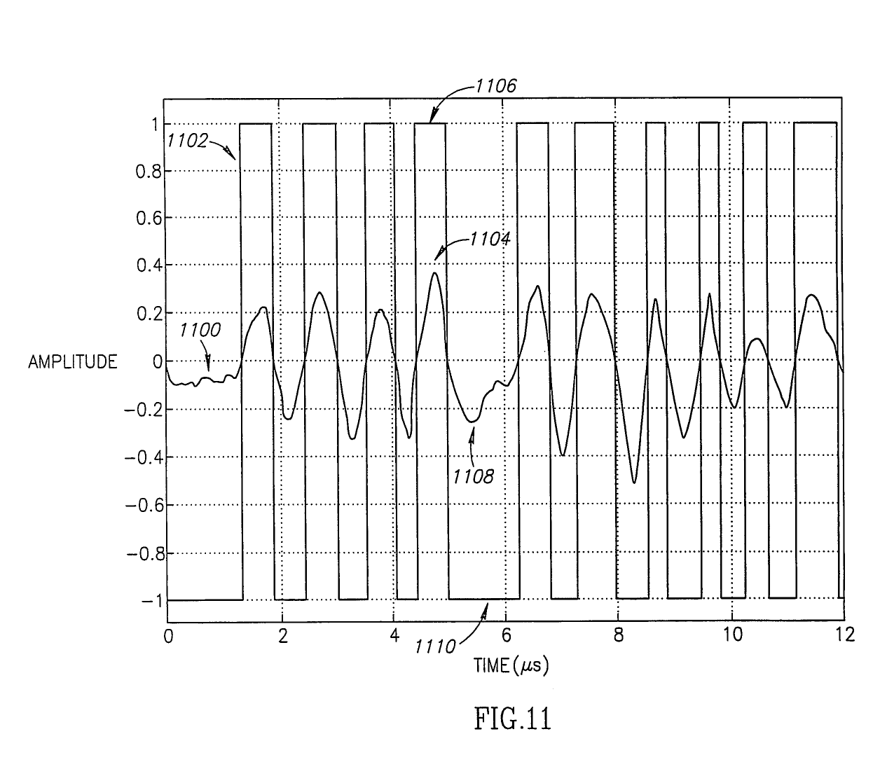

In block 822, the signal is time-reversed so that a time-reverse derived signal is created. In physics, time reversal is a signal processing technique that involves temporal reversing of a signal, so that it essentially “plays” or “flows” from end to start. Reference is now made to FIG. 10, which shows an exemplary time-reverse derived signal 1000. As shown, time-reverse derived signal 1000 is a temporal opposite of signal 900 of FIG. 9, from which it was derived. The dominant, strong amplitudes that appear in the range of 0-0.2 milliseconds of signal 900 (FIG. 9) are evident in the last 0.2 milliseconds of signal 1000, between 1.8 and 2 milliseconds. Time-reverse derived signal 1000 is referred to as an “exact” 824 time-reverse derived signal, since it has approximately the same structure as signal 900 (FIG. 9), but mirrored.

Optionally, a time-reverse derived signal is of a 1-bit 826 structure rather than an “exact” mirror of signal 900 (FIG. 9). A 1-bit signal has a non-sinusoidal, “square” waveform. A signal may be converted to a 1-bit signal before or after it is time-reversed. Let an “original” signal, either 900 (before time-reversal) or 1000 (after time-reversal), have a form A(t). Its values can be positive and/or negative. In reality, it is a modulated sinusoidal signal or a mixture of sinusoidal signals. After digitization, A(t) is a vector having positive and negative values and sometimes zero values. To obtain a 1-bit signal B(t) from A(t), one has to perform a transformation:

B(t)=+1 if A(t)>0; −1 if A(t)≦0

Reference is now made to FIG. 11, which graphically illustrates this transformation. An exemplary, 12 μs-long signal A(t) 1100 and its corresponding 1-bit signal B(t) 1102 are shown. Whenever A(t) 1100 has an amplitude higher than 0, it is transformed into a square, 1-bit form having a value of 1. For example, an amplitude 1104 of A(t) has a value of above 0, and is therefore transformed, in B(t) 1102, into a square form 1106. Similarly, whenever A(t) 1100 has an amplitude lower than 0, it is transformed into a square, 1-bit form having a value of −1. For example, an amplitude 1108 of A(t) has a value of below 0, and is therefore transformed, in B(t) 1102, into a square form 1110.

It should be noted, that the 1-bit transformation may be performed digitally, using a computer, or analogically, using analog electronic means.

Referring now back to FIG. 1, optionally, the time-reversing and/or the 1-bit signal deriving is performed by calibration controller 122.

Referring now to FIG. 8, in block 828, a time-reverse derived signal (such as time-reverse derived signal 1000 of FIG. 10) is optionally stored in a memory, along with information pertaining to the first and the second locations. Optionally, the memory is embedded within calibration controller 122 and/or essentially connected to it. Optionally, the memory is a portable memory device adapted to be read by the HIFU system and/or by a memory reader adapted to communicate with the HIFU system. For example, the portable memory device may be a flash card, a smart card, a Universal Serial Bus (USB) memory stick, a Compact Disc (CD) and/or the like.

In block 830, which may be performed instead of or in conjunction with the combination of blocks 822 and 828, the signal received by hydrophone 118 is stored in the memory. This signal may be time-reversed, and a calibration of the HIFU system may be finalized, at a later time. For example, the signal may be time-reversed shortly before performing a treatment using the HIFU system and/or when a HIFU system is deployed at a location in which it is used for performing treatment.

A Second Exemplary Calibration Method (Hereinafter “Backward Calibration”)

In an embodiment, a backward calibration of a HIFU system is performed, essentially using a calibration configuration 1200, by emitting a calibration ultrasonic signal from a first location within a tissue simulating medium, receiving the signal at a second location and deriving a time-reversed signal from the received signal. The time-reverse derived signal is stored in a memory, along with information pertaining to the relative position of the first and the second locations.

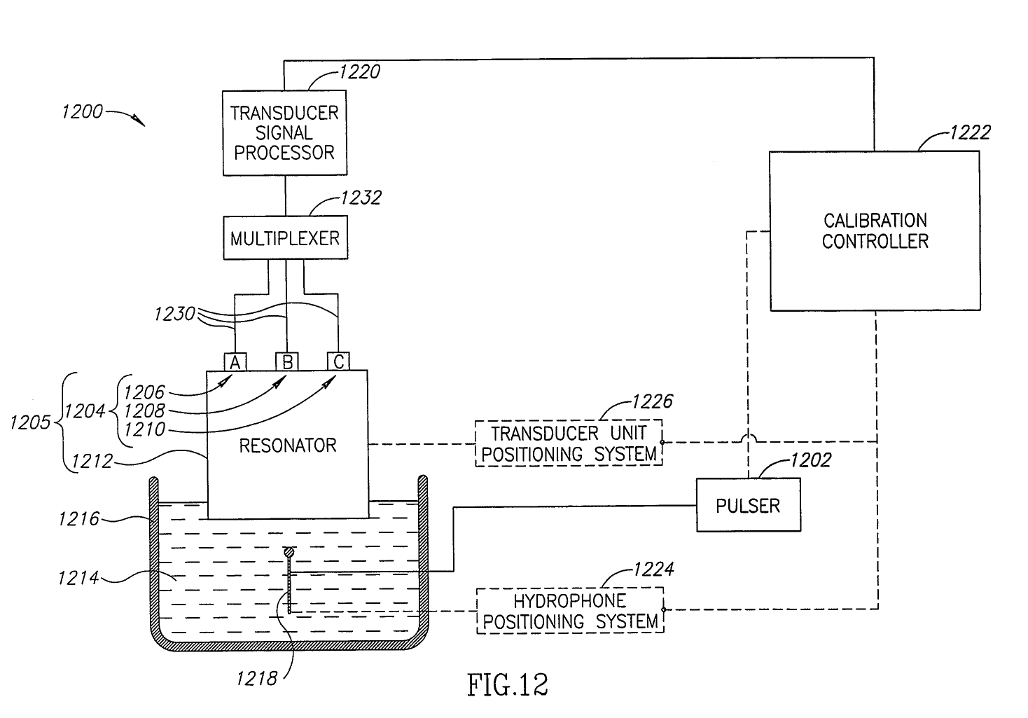

Reference is now made to FIG. 12, which shows an exemplary backward calibration configuration 1200. Backward calibration configuration 1200 optionally contains elements discussed in the forward calibration configuration 100 (FIG. 1) section. Persons of skill in the art will recognize that the following elements of backward calibration configuration 1200 may be essentially similar or identical to corresponding components discussed in regard to forward calibration configuration 100 (FIG. 1):
a pulser 1202 may be similar or identical to pulser 102;
a transducer set 1204 (which may include transducer A (1206), B (1208) and/or C (1210), may be similar or identical to transducer set 104;
a resonator 1212 may be similar or identical to resonator 112;
a tissue simulating medium 1214 may be similar or identical to tissue simulating medium 114, and may be optionally contained within a tank 1216 which may be similar or identical to tank 116;
a hydrophone 1218 may be similar or identical to hydrophone 118;
a transducer signal processor 1220 may be similar or identical to hydrophone signal processor 120;
a calibration controller 1222 may be similar or identical to calibration controller 122;
a hydrophone positioning system 1224 may be similar or identical to hydrophone positioning system 124;
a transducer unit positioning system 1226 may be similar or identical to transducer unit positioning system 126;

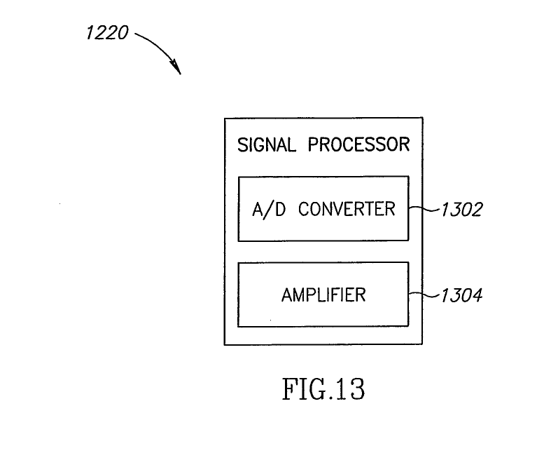

Some or all of the elements mentioned here may include some alterations in comparison to the way they are described in regard to forward calibration configuration 100 (FIG. 1):
Pulser 1202 may be adapted to selectively emit a pulse towards hydrophone 1218;
Hydrophone 1218 may be adapted to convert an electrical signal received from pulser 1202 to ultrasonic waves travelling in tissue simulating medium 1214;
Transducer signal processor 1220 may receive an electrical signal from a multiplexer 1232 and/or from transducers of transducer set 1204, the signal representing ultrasonic waves sensed by transducers of the transducer set. FIG. 13 shows a block diagram of transducer signal processor 1220, which may include an analog/digital (A/D) converter 1302 and an amplifier 1304. A/D converter 1302 is an electronic device adapted to convert electronic signals received from transducers of transducer set 1204 (FIG. 12) to digital data, so that the digital data represents ultrasonic waves sensed by the transducers. Amplifier 1304 is an electronic device adapted to amplify electronic signals received from transducers of transducer set 1204 (FIG. 12), so that these signals are more easily identified and treated.

Referring now to FIG. 12, multiplexer 1232, which may not be included in forward calibration configuration 100 (FIG. 1), is a device adapted to receive electrical signals over one or more channels 1230 from one or more transducers of transducer set 1204. Multiplexer 1232 may function as a switch, adapted to selectively connect a specific transducer to transducer signal processor 1220 and/or to calibration controller 1222. Multiplexer 1232 may alternately, based on a pre-defined sequence or based on user selection, switch between each individual transducer of transducer set 1204 and transducer signal processor 1220 and/or calibration controller 1222.

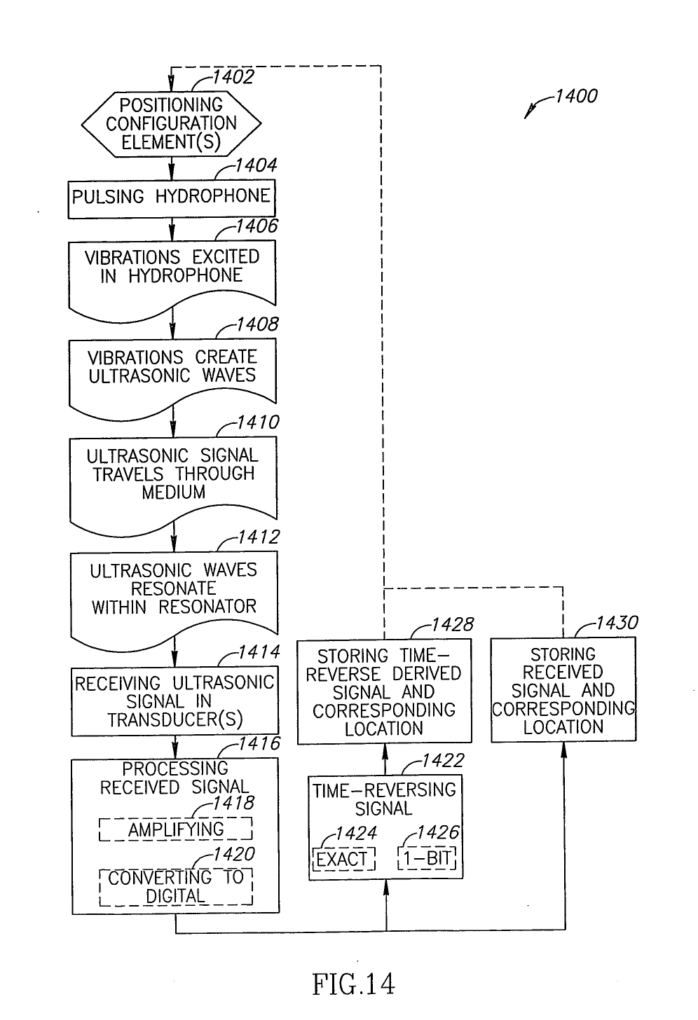

Reference is now made to FIG. 14, which shows a flow chart of the backward calibration 1400. Description of backward calibration 1400 is herein presented by referring to FIGS. 12 and 14 intermittently. Elements of FIG. 12 are notated by numerals ranging between 1200-1299, while elements of FIG. 14 are notated by numerals ranging between 1400-1499.

In a block 1402, at least one element of backward calibration configuration 1200 is positioned. For example, transducer unit positioning system 1226 may be employed to position transducer unit 1205. Yet another example is a positioning of hydrophone 1218 using hydrophone positioning system 1224. Calibration controller 1222 optionally keeps track of the element positioning(s), so that a relative position of resonator 1212 and/or transducer set 1204 (hereinafter “second location”) and hydrophone 1218 (hereinafter “first location”) is known. Optionally, the relative position is three-dimensional. Positioning of the at least one element(s) may be controlled by calibration controller 1222, which optionally stores information pertaining to the relative position of the first and the second locations and/or stores the first and the second locations themselves.

In a block 1404, pulser 1202 emits an electrical pulse, also referred to as a “calibration pulse”, towards hydrophone 1218. Optionally, the signal is approximately 0.1 μs (microseconds) long.

In a block 1406, the electrical pulse emitted by pulser 1202 excites vibrations in the pulsed hydrophone 1218. In a block 1408, the vibrations create ultrasonic waves.

In a block 1410, the ultrasonic waves travel from hydrophone 1218 within tissue simulating medium 1214. The ultrasonic waves (also “signal”) travel essentially as compression waves within tissue simulating medium 1214.

In a block 1412, the ultrasonic waves resonate within resonator 1212.

In a block 1414, the ultrasonic signal is received by a transducer of transducer set 1204. Optionally, the transducer of transducer set 1204 receives the signal over a pre-defined time window, so that a latter portion of the signal may not be received and/or be omitted after receiving. Optionally, the pre-defined period of time is inversely correlated to a resonant frequency of the relevant transducer. Usually, the higher the frequency, the shorter the pre-defined period is, and vice versa. For example, for a 1 MHz transducer, the pre-defined period may be 2 ms. For a 200 KHz transducer, the period may be five times longer, namely 10 ms. FIG. 9 shows a graphic representation of an exemplary ultrasonic signal 900 received by transducers of transducer set 1204. As shown, signal 900 has several dominant, strong amplitudes in the range of 0-0.2 milliseconds, after which the signal slowly fades. In the range of 0.8 milliseconds and 2 milliseconds, for example, amplitude changes in the signal are minimal, compared to the strong amplitudes demonstrated earlier. As the signal fades, the fading portion may become less and less useful for backward calibration 1400, and therefore, the time window may be pre-defined to omit this fading portion.

Optionally, steps of blocks 1402-1414 are repeated for any remaining one or more transducers of transducer set 1204, so that signals relating to each of the transducers are available for future use.

Optionally, multiple transducers of transducers set 1204 receive essentially the same ultrasonic signal emitted by hydrophone 1218. An electronic signal representing that ultrasonic signal may then be transmitted from each of the multiple transducers of transducers set 1204 to a multi-channel receiver (not shown), instead of to multiplexer 1232. The multi-channel receiver may be adapted to receive multiple distinct signals essentially simultaneously and to transfer them to one or more components of a block 1416.

In a block 1416, the signal may be processed. The signal is optionally amplified 1418 using amplifier 1304 (FIG. 13) and/or converted 1420 to a digital signal using A/D converter 1302 (FIG. 13). The amplification and/or the conversion may enable and/or support further manipulations to the signal. By way of example, converting the signal from analog to digital may allow its manipulation using a computer (such as calibration controller 1222), which essentially handles digital data.

Backward calibration 1400 optionally splits, so that either a block 1430 or a combination of blocks 1422 and a block 1428 is performed. Alternatively, both block 1430 and the combination of blocks 1422 and block 1428 may be performed.

In block 1422, the signal is time-reversed so that a time-reverse derived signal is created. Reference is now made to FIG. 10, which shows an exemplary time-reverse derived signal 1000. As shown, time-reverse derived signal 1000 is a temporal opposite of signal 900 of FIG. 9, from which it was derived. The dominant, strong amplitudes that appear in the range of 0-0.2 milliseconds of signal 900 (FIG. 9) are evident in the last 0.2 milliseconds of signal 1000, between 1.8 and 2 milliseconds. Time-reverse derived signal 1000 is referred to as an “exact” 1424 time-reverse derived signal, since it has approximately the same structure as signal 900 (FIG. 9), but mirrored.

Optionally, a time-reverse derived signal is of a 1-bit 1426 structure rather than an “exact” mirror of signal 900 (FIG. 9). A 1-bit signal has a non-sinusoidal, “square” waveform. A signal may be converted to a 1-bit signal before or after it is time-reversed. Let an “original” signal, either 900 (before time-reversal) or 1000 (after time-reversal), have a form A(t). Its values can be positive and/or negative. In reality, it is a modulated sinusoidal signal or a mixture of sinusoidal signals. After digitization, A(t) is a vector having positive and negative values and sometimes zero values. To obtain a 1-bit signal B(t) from A(t), one has to perform a transformation:

B(t)=+1 if A(t)≦0

Reference is now made to FIG. 11, which graphically illustrates this transformation. An exemplary, 12 μs-long signal A(t) 1100 and its corresponding 1-bit signal B(t) 1102 are shown. Whenever A(t) 1100 has an amplitude higher than 0, it is transformed into a square, 1-bit form having a value of 1. For example, an amplitude 1104 of A(t) has a value of above 0, and is therefore transformed, in B(t) 1102, into a square form 1106. Similarly, whenever A(t) 1100 has an amplitude lower than 0, it is transformed into a square, 1-bit form having a value of −1. For example, an amplitude 1108 of A(t) has a value of below 0, and is therefore transformed, in B(t) 1102, into a square form 1110.

It should be noted, that the 1-bit transformation may be performed digitally, using a computer, or analogically, using analog electronic means.

Optionally, the time-reversing and/or the 1-bit signal deriving are performed by calibration controller 1222.

Referring now to FIG. 14, in block 1428, a time-reverse derived signal (such as time-reverse derived signal 1000 of FIG. 10) is optionally stored in a memory, along with information pertaining to the first and the second locations. Optionally, the memory is embedded within calibration controller 122 and/or essentially connected to it. Optionally, the memory is a portable memory device adapted to be read by the HIFU system and/or by a memory reader adapted to communicate with the HIFU system. For example, the portable memory device may be a flash card, a smart card, a Universal Serial Bus (USB) memory stick, a Compact Disc (CD) and/or the like.

In block 1430, which may be performed instead of or in conjunction with the combination of blocks 1422 and 1428, the signal received by transducers of transducer set 1204 is stored in the memory. This signal may be time-reversed, and a calibration of the HIFU system may be finalized, at a later time. For example, the signal may be time-reversed shortly before performing a treatment using the HIFU system and/or when a HIFU system is deployed at a location in which it is used for performing treatment.

HIFU System Usage, User Interface and Body Contouring In calibration methods 800 (FIG. 8) and 1400 (FIG. 14) described above, ultrasonic signals are stored in a memory along with information pertaining to a relative location of the point from which the signal is emitted (referred to as “first location”) and the point in which the signal is received (referred to as “second location”). These data stored in the memory may be used later to focus an ultrasonic signal on soft tissues, such as adipose tissue, whose destruction is desired. Since the memory includes data that enables emitting a certain time-reverse derived ultrasonic signal for a given relative location, a user interface may allow a user to select a relative location of soft tissue and one or more transducers of a treatment HIFU system, so that a signal emitted by the HIFU system is focused essentially on the soft tissue. Optionally, the time-reverse derived signal is emitted using digital output circuitry of the HIFU system.

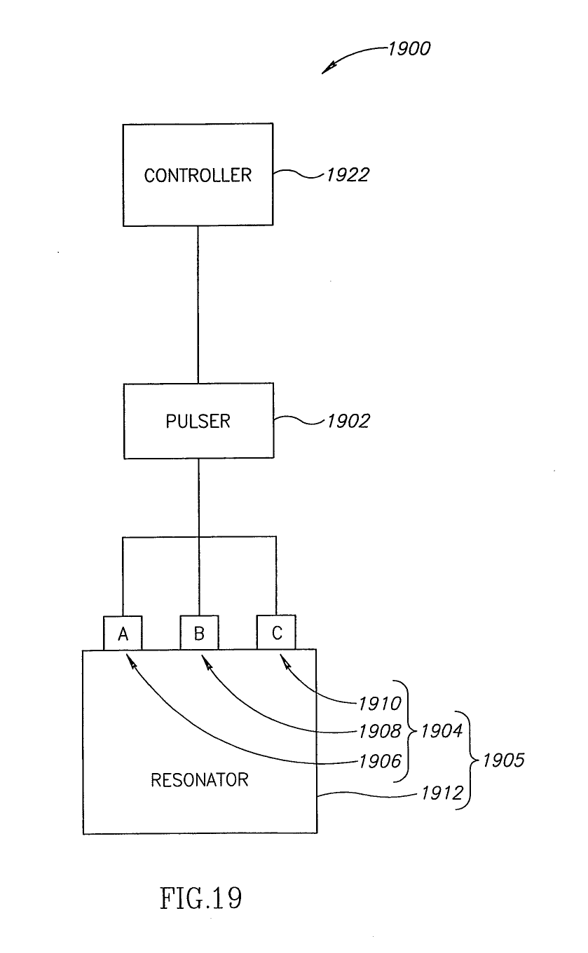

Reference is now made to FIG. 19, which shows a block diagram of a HIFU system 1900. HIFU system 1900 may include a controller 1922, a pulser 1902, a transducer set 1904 including one or more transducers (such as transducers A, B and C, 1906, 1908 and 1910, respectively) and a resonator 1912. Transducer set 1904 and resonator 1912 may be jointly referred to as a transducer unit 1905. One or more elements of HIFU system 1900 may be elements used in forward calibration configuration 100 (FIG. 1) and/or in calibration configuration 1200 (FIG. 12), so that treatment is essentially performed accurately, corresponding to the preceding calibration. For example, transducer unit 1905 may be the same transducer unit 105 used in forward calibration configuration 100.

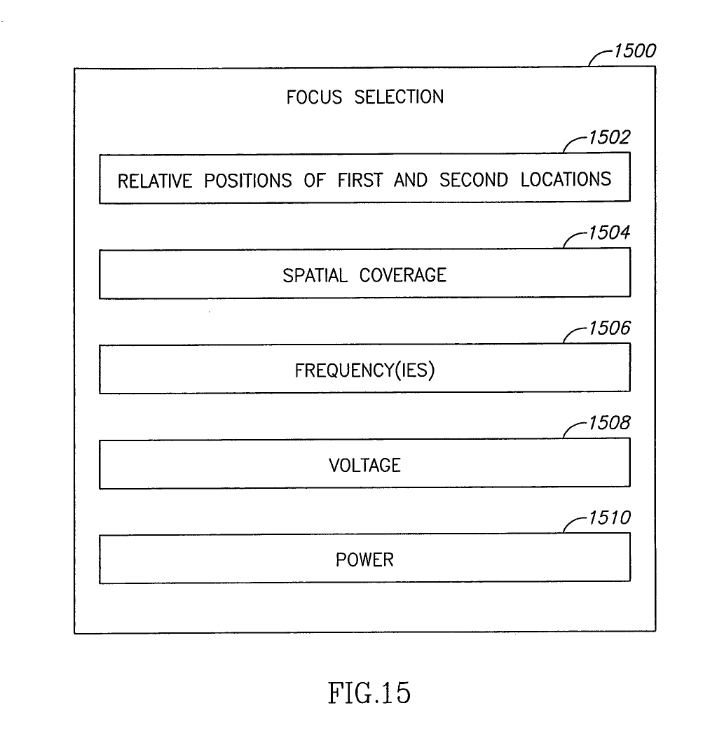

Controller 1922 may control the emitting of signals by pulser 1902 and/or by transducer unit 1905. Controller 1922 may include a user interface (shown in FIG. 15 and explained below) for allowing a caregiver to control HIFU system 1900.

User Interface and Usage

In an embodiment, the HIFU system comprises a user interface adapted to allow selection of at least one focus parameter which is based on at least one calibration of the HIFU system. Reference is now made to FIG. 15, which shows an exemplary Graphical User Interface (GUI) 1500 for such selection. GUI 1500 may include user-exercisable options, such as an option for selecting a relative position of the first and the second locations (this option hereinafter referred to as “treatment node position”) 1502, an option for selecting spatial coverage (this option hereinafter referred to as “treatment coverage”) 1504, an option for selecting frequency(ies) (this option hereinafter referred to as “treatment frequencies”) 1506, an option for selecting excitation voltage amplitude (this option hereinafter referred to as “treatment voltage”) 1508 and/or an option for selecting a power level (this option hereinafter referred to as “treatment power”) 1510.

Optionally, treatment node position 1502 is a relative position of a first location from which a time-reverse derived ultrasonic signal is emitted and a second location of soft tissues. By way of example, a user may use treatment node position 1502 to select a location of a focal area within the soft tissue, on which a focusing on ultrasonic signals is desired.

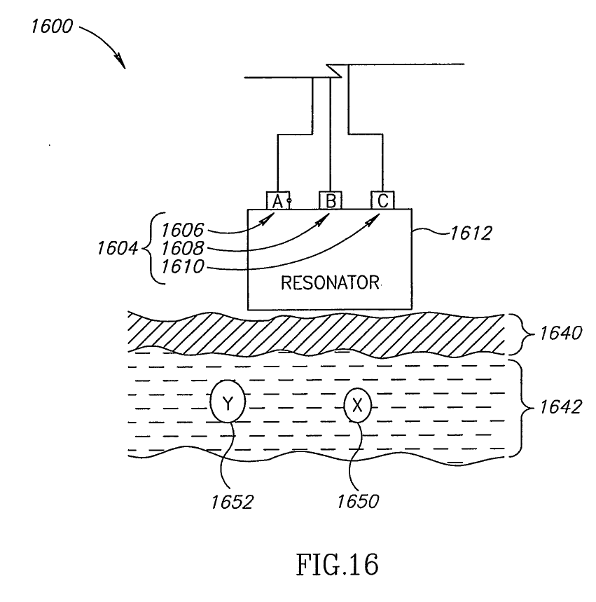

Reference is now made to FIG. 16, which shows a cross section view of in-vivo tissue destruction using a HIFU system 1600. For simplicity of presentation, HIFU system 1600 is presented only with its resonator 1612 and its transducer set 1604 which may include transducers A 1606, B 1608 and/or C 1610. A skin layer 1640 is essentially covering soft tissues 1642. A three-dimensional focal area X 1650 within soft tissues 1642 is an area on which focusing of ultrasonic signals is desired.

Referring now to FIGS. 15 and 16 interchangeably, a user may use treatment node position 1502 to focus an ultrasonic signal on focal area X 1650 by entering a location of the focal area into GUI 1500. Furthermore, a user may destroy soft tissue in multiple focal areas essentially simultaneously and/or sequentially. For example, by entering locations of focal area X 1650 and of a focal area Y 1652, the HIFU system may simultaneously emit multiple ultrasonic signals, optionally from separate transducers, while some of the signals are focused on focal area X and some on focal area Y. Additionally or alternatively, the HIFU system may emit multiple ultrasonic signals essentially sequentially, optionally from separate transducers, while some of the signals are focused on focal area X and some on focal area Y.

Optionally, treatment coverage 1504 is a spatial coverage of a time-reverse derived ultrasonic signal emitted from HIFU system 1600. Treatment coverage 1504 may define a three-dimensional shape of focal area X 1650. Treatment of a three-dimensional focal area such as focal area X 1650 may be performed by using one or more transducers of transducer set 1604 to emit ultrasonic signals towards one or more areas forming, together, the focal area.

Optionally, treatment frequencies 1506 is a frequency characteristic of a time-reverse derived ultrasonic signal emitted from the HIFU system. Each transducer of different resonant frequency—which is the frequency of ultrasonic signals the transducer emits when it is pulsed with electrical signals.

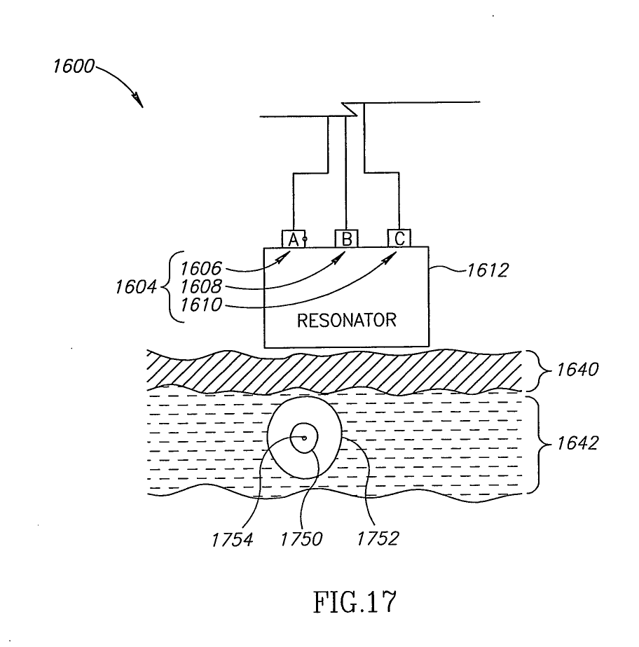

Optionally, two or more of transducers A 1606, B 1608, C 1610 and/or other transducer(s) that may exist, have a different resonant frequency. Reference is now made to FIG. 17, which shows HIFU system 1600, wherein at least two ultrasonic signals emitted from the HIFU system have different frequency characteristics. For example, two ultrasonic signals may be focused on a focal point 1754, located within soft tissues 1642. Commonly, an ultrasonic signal focused on a certain focal point is able to destroy soft tissue in a three-dimensional area (a “focal area”) surrounding the focal point. Generally, the higher the frequency of the signal, the smaller the focal area is. A first ultrasonic signal which is focused on focal point 1754 may have a focal area 1750. A second ultrasonic signal which is focused on focal point 1754 may have a focal area 1752, which is essentially larger than focal area 1750 of the first ultrasonic signal. That is, because a frequency of the second ultrasonic signal is lower than that of the first ultrasonic signal.

For example, the first ultrasonic signal may have a frequency of 1 MHz (1000 KHz), while the second ultrasonic signal may have a relatively close frequency of 900 KHz. The two ultrasonic signals may temporally overlap; they may either both be emitted essentially simultaneously, or one may be emitted while the other one is still resonating. When the two ultrasonic signals that are focused on the same focal point 1750 temporally overlap, collision and/or interaction of their ultrasonic waves in and around the focal point may behave according to a phenomena often referred to as “parametric excitation”. The interacting ultrasonic waves may cause an increase in treatment area (which is optionally larger than focal area 1752) and/or enhance the efficacy of cavitation, thereby yielding enhanced soft tissue destruction.

As another example, the first ultrasonic signal may have a frequency of 1 MHz (1000 KHz), while the second ultrasonic signal may have a relatively distant frequency of 200 KHz. Temporal overlapping of these signals may improve efficacy of cavitation, thereby yielding enhanced soft tissue destruction.

Optionally, treatment voltage 1508 is an excitation voltage amplitude with which one or more transducers of transducer set 1604 is pulsed. Each transducer of transducer set 1604, such as transducer A 1606, B 1608 or C 1610, may be pulsed using a same or a different voltage amplitude. Different voltage amplitudes applied to a transducer may influence acoustic characteristics of its output.

Optionally, treatment power 1510 is power level with which one or more transducers of transducer set 1604 is pulsed. Treatment power 1510 may be defined in watts. Each transducer of transducer set 1604, such as transducer A 1606, B 1608 or C 1610, may be pulsed using a same or a different power level. Different power levels applied to a transducer may influence acoustic characteristics of its output.

In an embodiment, GUI 1500 may allow selection of one or more targeting profile(s). The targeting profile may include a pre-defined combination of one or more settings of treatment node position 1502, treatment coverage 1504, treatment frequencies 1506, treatment voltage 1508 and/or treatment power 1510. Optionally, a user may define the combination and/or different settings associated with treatment node position 1502, treatment coverage 1504, treatment frequencies 1506, treatment voltage 1508 and/or treatment power 1510.

Body Cntouring

In an embodiment, the HIFU system is used in a body contouring procedure—a procedure wherein adipose tissues are destroyed for reshaping and essentially enhancing the appearance of a human body.

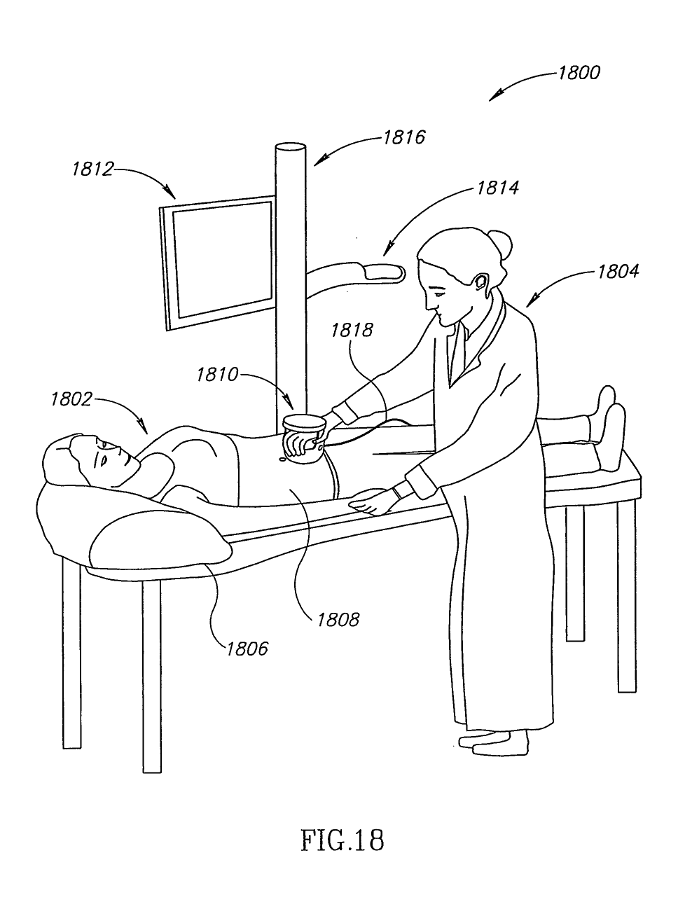

Reference is now made to FIG. 18, which shows an exemplary treatment 1800 of a patient 1802 by a caregiver 1804. Caregiver 1804 may be, for example, a physician, a nurse and/or any other person legally and/or physically competent to perform a body contouring procedure involving non-invasive adipose tissue destruction. Patient 1802 optionally lies on a bed 1806 throughout treatment 1800.

Caregiver 1804 may hold a transducer unit 1810 against an area of patient's 1802 body wherein destruction of adipose tissues is desired. For example, transducer unit 1810 may be held against the patient's 1802 abdomen 1808. Transducer unit 1810 may comprise one or more transducers (not shown) and/or one of more resonators (not shown). Transducer unit 1810 may be connected by at least one wire 1818 to a controller (not shown) and/or to a power source (not shown).

Optionally, a user interface 1500 (FIG. 15) is displayed on a monitor 1812, which may be functionally affixed to a rack, such as pillar 1816. A transducer unit 1810 storage ledge 1814 may be provided on pillar 1816 or elsewhere.

Body contouring may be performed by emitting one or more ultrasonic pulses from transducer unit 1810 while it is held against a certain area of the patient's 1802 body. Then, transducer unit 1810 is optionally re-positioned above one or more additional areas and the emitting is repeated. Each position of transducer unit 1810 may be referred to as a “node”. A single body contouring treatment may include treating a plurality of nodes.

In the description and claims of the application, each of the words “comprise” “include” and “have”, and forms thereof, are not necessarily limited to members in a list with which the words may be associated.

The invention has been described using various detailed descriptions of embodiments thereof that are provided by way of example and are not intended to limit the scope of the invention. The described embodiments may comprise different features, not all of which are required in all embodiments of the invention. Some embodiments of the invention utilize only some of the features or possible combinations of the features. Variations of embodiments of the invention that are described and embodiments of the invention comprising different combinations of features noted in the described embodiments will occur to persons with skill in the art. It is intended that the scope of the invention be limited only by the claims and that the claims be interpreted to include all such variations and combinations.

What is claimed is:

1. A method for creating a time reversed signal adapted to destroy a soft tissue, the method comprising:
emitting a first ultrasonic signal from a transmitter towards a tissue simulating medium which simulates said soft tissue, wherein the first ultrasonic signal has a first frequency characteristic;
receiving the first ultrasonic signal in a receiver and converting the first ultrasonic signal to an electrical signal;
converting the electrical signal to a digital signal; and
time-reversing the digital signal to produce the time-reversed signal.

2. The method according to claim 1, wherein the transmitter is a transducer unit comprising at least one transducer attached to at least one resonator, and the receiver is a sensor embedded in the tissue simulating medium.

3. The method according to claim 2, further comprising:
emitting a second ultrasonic signal from the transducer unit towards said tissue simulating medium, wherein the second ultrasonic signal has a second frequency characteristic which is different than the first frequency characteristic of the first ultrasonic signal;
receiving the second ultrasonic signal in the receiver and converting the second ultrasonic signal to a second electrical signal;
converting the second electrical signal to a second digital signal; and
time-reversing the second digital signal to produce a second time-reversed signal.

4. The method according to claim 2, further comprising:
emitting a second ultrasonic signal from a second transducer unit towards the tissue simulating medium, wherein the second ultrasonic signal has a second frequency characteristic which is different than the first frequency characteristic of the first ultrasonic signal;
receiving the second ultrasonic signal in the receiver and converting the second ultrasonic signal to a second electrical signal;
converting the second electrical signal to a second digital signal; and
time-reversing the second digital signal to produce a second time-reversed signal.

5. The method according to claim 1, wherein the transmitter is a sensor embedded in the tissue simulating medium and the receiver is a transducer unit comprising at least one transducer attached to at least one resonator.

6. The method according to claim 5, further comprising:
emitting a second ultrasonic signal from the sensor towards the tissue simulating medium, wherein the second ultrasonic signal has a second frequency characteristic which is different than the first frequency characteristic of the first ultrasonic signal;
receiving the second ultrasonic signal in the transducer unit and converting the second ultrasonic signal to a second electrical signal;
converting the second electrical signal to a second digital signal; and
time-reversing the second digital signal to produce a second time-reversed signal.

7. The method according to claim 5, further comprising:
emitting a second ultrasonic signal from the sensor towards the tissue simulating medium, wherein the second ultrasonic signal has a second frequency characteristic which is different than the first frequency characteristic of the first ultrasonic signal;
receiving the second ultrasonic signal in a second transducer unit and converting the second ultrasonic signal to a second electrical signal;
converting the second electrical signal to a second digital signal; and
time-reversing the second digital signal to produce a second time-reversed signal.

8. The method according to claim 1, wherein the soft tissue is an adipose tissue.

9. The method according to claim 1, further comprising storing the time-reversed signal in a memory, along with a corresponding datum pertaining to a relative location of the transmitter and the receiver.

10. The method according to claim 1, further comprising converting the digital signal to a 1-bit signal.

11. The method according to claim 1, further comprising converting the time-reversed signal to a 1-bit signal.

12. A system adapted to produce a time-reversed signal for destroying a soft tissue, the system comprising:
a transmitter adapted to emit an ultrasonic signal towards a tissue simulating medium which simulates the soft tissue;
a receiver adapted to receive said ultrasonic signal and to convert said ultrasonic signal to an electrical signal;
an analog-to-digital converter adapted to convert said electrical signal to a digital signal; and
a signal processor adapted to time-reverse said digital signal and to produce said time-reversed signal.

13. The system according to claim 12, wherein said transmitter is a transducer unit comprising at least one transducer attached to at least one resonator, and said receiver is a sensor embedded in said tissue simulating medium.

14. The system according to claim 13, wherein said at least one transducer comprises two or more transducers, each having a different resonant frequency.

15. The system according to claim 12, wherein said transmitter is a sensor embedded in said tissue simulating medium and said receiver is a transducer unit comprising at least one transducer attached to at least one resonator.

16. The system according to claim 15, wherein said at least one transducer comprises two or more transducers, each having a different resonant frequency.

17. The system according to claim 12, wherein the soft tissue is an adipose tissue.

18. The system according to claim 12, further comprising a memory adapted to store said time-reverse derived signal, along with a corresponding datum pertaining to a relative location of the transmitter and the receiver.

19. The system according to claim 12, wherein said time-reversed signal is a 1-bit signal.

20. A method for, destroying a soft tissue within a focal area, the method comprising emitting a first time-reverse derived ultrasonic signal focused on a focal point within the focal area, wherein said time-reverse derived ultrasonic signal has a first frequency characteristic.

21. The method according to claim 20, wherein the first time-reverse derived ultrasonic signal is adapted to induce cavitation within the focal area.

22. The method according to claim 20, wherein the first time-reverse derived ultrasonic signal corresponds to a signal emitted by a transducer and received by a sensor embedded in a tissue simulating medium.

23. The method according to claim 20, wherein the first time-reverse derived ultrasonic signal corresponds to a signal emitted by a sensor embedded in a tissue simulating medium and received by a transducer.

24. The method according to claim 20, wherein the soft tissue is an adipose tissue.

25. The method according to claim 20, wherein the first time-reverse derived ultrasonic signal is based on a 1-bit signal.

26. The method according to claim 20, further comprising:
emitting a second time-reverse derived ultrasonic signal which temporally overlaps the first time-reverse derived ultrasonic signal and is focused on the focal point, wherein the second time-reverse derived ultrasonic signal has a second frequency characteristic which is different than the first frequency characteristic of the first time-reverse derived ultrasonic signal.

27. A device adapted to destroy a soft tissue within a focal area, the device comprising a transducer unit adapted to emit a first time-reverse derived ultrasonic signal having a first frequency characteristic, wherein the first time-reverse derived ultrasonic signal is adapted to be focused on a focal point within said focal area.

28. The device according to claim 27, wherein the soft tissue is an adipose tissue.

29. The device according to claim 27, wherein the first time-reverse derived ultrasonic signal corresponds to a signal received by a sensor embedded in a tissue simulating medium which simulates the soft tissue.

30. The device according to claim 27, wherein the first time-reverse derived ultrasonic signal corresponds to a signal received by a transducer.

31. The device according to claim 27, wherein the first time-reverse derived ultrasonic signal is adapted to induce cavitation within the focal area.

32. The device according to claim 27, further comprising an interface module adapted to interface with a memory and to retrieve a digital representation of the first time-reverse derived ultrasonic signal stored in the memory.

33. The device according to claim 27, wherein the time-reverse derived ultrasonic signal is based on a 1-bit signal.

34. The device according to claim 27, wherein:
the transducer unit is adapted to emit a second time-reverse derived ultrasonic signal which temporally overlaps the first time-reverse derived ultrasonic signal;
the second time-reverse derived ultrasonic signal is focused on the focal point; and
the second time-reverse derived ultrasonic signal has a second frequency characteristic which is different than the first frequency characteristic of the first time-reverse derived ultrasonic signal.

35. The device according to claim 27, further comprising a second transducer unit adapted to emit a second time-reverse derived ultrasonic signal which temporally overlaps the first time-reverse derived ultrasonic signal,
wherein the second time-reverse derived ultrasonic signal is focused on the focal point, and
wherein the second time-reverse derived ultrasonic signal has a second frequency characteristic which is different than the first frequency characteristic of the first time-reverse derived ultrasonic signal.

36. A non-volatile memory device adapted to be read by a soft tissue destruction device, comprising:
a first time-reverse derived ultrasonic signal having a first frequency characteristic; and
a datum pertaining to a relative location of a transmitter and a receiver, wherein the datum corresponds to said first time-reverse derived ultrasonic signal.

37. The memory device according to claim 36, wherein said first time-reverse derived ultrasonic signal is a 1-bit signal.

38. The memory device according to claim 36, further comprising a second time-reverse derived ultrasonic signal having a second frequency characteristic which is different than said first frequency characteristic of said first time-reverse derived ultrasonic signal.

39. A user interface adapted to control a soft tissue destruction device, the user interface comprising a user-selectable ultrasonic focus parameter pertaining to a relative position of a transducer unit and a focal point within the soft tissue.

40. The user interface according to claim 39, further comprising a second ultrasonic focus parameter pertaining to a spatial coverage of at least one time-reverse derived ultrasonic signal adapted to be emitted from the soft tissue destruction device.

41. The user interface according to claim 39, further comprising a second ultrasonic focus parameter pertaining to at least one frequency value of a time-reverse derived ultrasonic signal adapted to be emitted from the soft tissue destruction device.

42. The user interface according to claim 39, fuirther comprising a second ultrasonic focus parameter pertaining to at least two frequency values of corresponding at least two time-reverse derived ultrasonic signals adapted to be emitted from the soft tissue destruction device.

43. The user interface according to claim 39, further comprising a second ultrasonic focus parameter pertaining to a voltage amplitude adapted to excite a transducer of the soft tissue destruction device.

44. The user interface according to claim 39, further comprising a second ultrasonic focus parameter pertaining to a power level adapted to excite a transducer of the soft
